(19) United States
(12) Patent Application Publication
(10) Pub. No.: US 2020/0323882 A1
(43) Pub. Date: Oct. 15, 2020

(54) PHLIP®-MEDIATED TARGETING OF CORTICOSTEROIDS TO DISEASED TISSUE

(71) Applicants: University of Rhode Island Board of Trustees, Kingston, RI (US); Yale University, New Haven, CT (US)

(72) Inventors: Yana K. Reshetnyak, Saunderstown, RI (US); David Wolfsohn, Easton, CT (US); Anna Moshnikova, Warwick, RI (US); Oleg A. Andreev, Saunderstown, RI (US); Donald M. Engelman, New Haven, CT (US)

(21) Appl. No.: 16/818,527

(22) Filed: Mar. 13, 2020

Related U.S. Application Data
(60) Provisional application No. 62/819,090, filed on Mar. 15, 2019.

(51) Int. Cl. A61K 31/573; A61K 47/64; A61K 47/34; A61K 9/00; A61P 35/00; A61P 17/00; A61P 19/02; A61P 37/02; A61P 11/00

(52) U.S. Cl. CPC A61K 31/573; A61K 47/64; A61K 47/34; A61K 9/0019; A61P 11/00; A61P 35/00; A61P 17/00; A61P 19/02; A61P 37/02; A61K 9/0014

(57) ABSTRACT

The invention features a composition comprising a corticosteroid and a pHLIP® peptide. pHLIP® peptides target corticosteroids to cell surface acidity in inflamed and fibrotic tissues, where they translocate corticosteroids across plasma membranes into the cytoplasms of cells, thereby suppressing inflammation in the targeted tissues while avoiding the side effects resulting from non-targeted administration.

### RELATED APPLICATIONS

This application claims the benefit of priority under 35 U.S.C. § 119(e) to U.S. Provisional Application No. 62/819,090, filed Mar. 15, 2019, the entire contents of which is incorporated herein by reference in its entirety.

### STATEMENT AS TO FEDERALLY SPONSORED RESEARCH

This invention was made with government support under Grant No. R01 GM073857 awarded by the National Institute of Health. The government has certain rights in the invention.

### INCORPORATION BY REFERENCE OF SEQUENCE LISTING

The contents of the sequence listing text file named “40984-515001WO_Sequence_Listing_ST25.txt”, which was created on Apr. 27, 2020 and is 98,304 bytes in size, is hereby incorporated by reference in its entirety.

### FIELD OF THE INVENTION

The present invention relates to corticosteroid targeted therapy.

### BACKGROUND

Corticosteroids are a class of synthetic analogs of steroid hormones that are produced in the adrenal cortex. Synthetic corticosteroids are effective in the management of a variety of disease states including severe inflammatory responses, autoimmune disorders, and neoplasia. However systemic and non-targeted use of corticosteroids is associated with serious side effects. Common complications associated with corticosteroid administration include rapid development of resistance in addition to immunosuppression (susceptibility to septic complications). Each of these confounding disadvantages can restrict the duration of administration and limit the successful resolution of aggressive or advanced conditions. Use of corticosteroids can cause adverse side effects such as weight gain, edema, insomnia, acne, hypertension, diabetes, metabolic syndrome, cataracts, immunosuppression, impaired wound healing, osteoporosis, growth retardation, myalgias, and adrenal insufficiency. Moreover, systemic suppression of the immune system can increase the risk of infection.

### SUMMARY OF THE INVENTION

The invention provides a solution to the limitations and drawbacks of clinical use of corticosteroids. Targeted approaches that predominantly deliver corticosteroids to inflamed tissue as described herein reduces and/or avoids systemic immunosuppression and other off-target effects, greatly improving the effective uses of corticosteroids. The invention provides a means for specific targeting and intracellular delivery of corticosteroids to cells in acidic diseased tissues. Targeted delivery predominantly affects the targeted tissue and reduces the exposure of healthy tissue to corticosteroids, thereby conferring clinical benefits while reducing systemic immunosuppression and other serious side effects.

Accordingly, the invention features a composition comprising a corticosteroid compound and a pHLIP® peptide. In some examples, the corticosteroid is a synthetically produced molecule. In some examples, the corticosteroid is 2,000 Dalton in mass or less, e.g., less than 1,500 Daltons, less than 1,000 Daltons, less than 500 Daltons, less than 400 Daltons, less than 300 Dalton in mass. For example, Dexamethasone has an average molecular mass of 392.461 Dalton.

The invention provides a solution to the side effect problem, because pHLIP® peptide sequences mediate targeting of the surface acidity in diseased tissue cells, without targeting normal tissues. In the diseased tissue, specific delivery of the potent corticosteroids occurs by translocation directly across the plasma membrane (bypassing endocytotic uptake) into the cytoplasms of the targeted cells, where the therapeutic targets are present. In this way, a corticosteroid together with a pHLIP® peptide can provide targeted therapy.

In preferred embodiments, the cargo compound, e.g., a corticosteroid, is preferentially targeted to inflamed tissue. The composition mediates entry of the cargo compound, e.g., a corticosteroid compound, such that the corticosteroid suppresses inflammation and immune reaction locally in the diseased tissue, e.g., the corticosteroid as delivered to cells by the pHLIP® peptide leads to limited preferential targeting of inflamed tissues. By preferential targeting is meant using a pHLIP® peptide to bind to a cell and deliver its cargo in a diseased tissue is least 10%, 20% 30%, 40% 50% 75%, 2-fold, 3-fold, 5-fold, 7-fold, 10-fold or more compared to the level of entry of the cargo into a cell/tissue comprising a normal or basic pH. For example, the cargo is a corticosteroid.

Exemplary corticosteroids include dexamethasone, betamethasone or derivatives thereof as well as others described below. In some examples, the composition (construct) further comprises a linker between said corticosteroid and said pHLIP® peptide. An exemplary linker comprises a disulfide bond, or an acid-liable bond, or an ester bond as a connection to the corticosteroid cargo molecule. In some examples, the linker is cleavable; alternatively, the linker is not cleavable. In embodiments, the linker is a polyethylene glycol (PEG) polymer. For example, the linker comprising the PEG polymer may comprise from 2 to 24 PEG units.

Modulators are optionally present in the construct/structure to change the polarity of the composition. For example, the compositions further comprises a polar modulator.

The compositions include a pHLIP® peptide, which confers the function of targeting to acidic vs. non-acidic cells or tissues. For example, the composition includes a pHLIP® peptide comprising the sequence ADDQNPWRAYLDLLFPTDTLLLDLLWXA (SEQ ID NO: 1) or ADQDNPWRAYLDLLFPTDTLLLDLLWXA (SEQ ID NO: 2), wherein upper case “X” indicates any amino acid residue and can include lysine (Lys), Cysteine (Cys), or Azido-containing amino acid. An exemplary composition comprises the following structure:

A-L-B

wherein “A” is a first pHLIP® peptide comprising the sequence ADDQNPWRAYLDLLFPTDTLLLDLLWXA (SEQ ID NO: 1) or ADQDNPWRAYLDLLFPTDTLLLDLLWXA (SEQ ID NO: 2), “B” is a second pHLIP® peptide comprising the sequence ADDQNPWRAYLDLLFPTDTLLLDLLWXA (SEQ ID NO: 1) or ADQDNPWRAYLDLLFPTDTLLLDLLWXA (SEQ ID NO: 2),

wherein upper case “X” indicates any amino acid residue and can include lysine (Lys), Cysteine (Cys), or Azido-containing amino acid; “L” is a polyethylene glycol linker, and each “-” is a covalent bond.

In some examples, the composition described herein has the following structure: A-L-Cs, wherein “A” is a pHLIP® peptide, “L” is a polyethylene glycol linker; “Cs” or “CS” is a corticosteroid, and wherein each “-” is a covalent bond.

Also within the invention are methods of treatment of undesirable or pathological conditions. For example, a method of local immunosuppression is carried out by administering to a subject a composition comprising a corticosteroid and a pHLIP® peptide. The subject may comprise an inflamed and fibrotic tissue, e.g., an asthma, arthritis, reactive airway diseases, chronic obstructive pulmonary disease (COPD), pneumonitis, sarcoidosis, hepatitis, nephritis, chronic kidney diseases (CKD), dermatitis, hives, angioedema, psoriasis, optic inflammation, nasal polyps, pemphigus vulgaris, lupus erythematosus, atherosclerosis, nonalcoholic fatty liver disease (NAFLD), nonalcoholic steatohepatitis (NASH), colitis, Crohn's disease, hepatitis, enteritis, polymyositis, leukemia, lymphoma, synovitis, tendonitis, or cerebral edema. In some examples, the composition is systemically administered. In other examples, the composition is injected directly into a diseased tissue or topically applied. The composition may also be systemically administered. Optionally, the corticosteroid is delivered into the cytosols of macrophages. The corticosteroid is targeted to inflamed tissue to induce a biological effect predominantly within the inflamed tissue. Preferably, the corticosteroid is delivered intracellularly to induce a biological effect.

Advantages of the compositions and methods described herein include pHLIP® peptide-mediated corticosteroid delivery preferentially to a diseased tissue or cell, thereby minimizing systemic immunosuppression and reduces side effects. The pHLIP® peptide element of the construct/structure is critical to the preferential targeting capability, i.e., limited inflamed tissue targeting ability occurs in the absence of said pHLIP®

As described above, the composition optionally comprises a linker between the corticosteroid and said pHLIP® peptide. Exemplary linkers include a disulfide bond, or an acid-labile bond, or an ester bond. In some examples, the linker is cleavable. In other examples, the linker is not cleavable. Exemplary linkers include those that are self-immolating (“self-immolative”). Examples include linkers with a disulfide bond and ester bond that are cleaved after delivery. Self-immolative elimination is a spontaneous and irreversible disassembly of a multicomponent compound into its constituent fragments through a cascade of electronic elimination processes. Self-immolative elimination is driven by an increase in entropy coupled with the irreversible formation of thermodynamically stable products (e.g. CO₂). In examples, the linker is a polyethylene glycol (PEG) polymer. For example, the linker comprising the PEG polymer may comprise from 2 to 24 PEG units.

A modulator of polarity is optionally included in the composition. Such a polar modulator increases the overall polarity of the construct. The polar modulator will decrease Log P of [cargo-modulator] (Log P<2.5). Non-limiting examples of modulators are PEG polymers, cyclic polar peptides. A modulator is added to make the composition more polar. Such polar modulators have an advantage in improving the solubility of the construct and/or the targeting of diseased tissues relative to normal tissues.

In some examples, the composition comprises 2 or more pHLIP® peptides. Exemplary constructs comprise the following structure: A-L-B, in which

Wherein “A” is a first pHLIP® peptide comprising the sequence ADDQNPWRAYLDLLFPTDTLLLDLLWXA (SEQ ID NO: 1) or ADQDNPWRAYLDLLFPTDTLLLDLLWXA (SEQ ID NO: 2), “B” is a second pHLIP® peptide comprising the sequence ADDQNPWRAYLDLLFPTDTLLLDLLWXA (SEQ ID NO: 1, or ADQDNPWRAYLDLLFPTDTLLLDLLWXA (SEQ ID NO: 2) wherein upper case “X” indicates any amino acid residue and can include lysine (Lys), Cysteine (Cys), or Azido-containing amino acid; and “L” is a polyethylene glycol linker, and each “-” is a covalent bond.

Also within the invention is a method of treatment of inflamed and fibrotic tissues comprising the administration of a composition comprising a corticosteroid and a pHLIP® peptide to a subject as described above as a monotherapy or in combination with other anti-inflammatory or anti-fibrotic therapies. For example, the subject is diagnosed with a disease for which use of a corticosteroid is indicated, including asthma, arthritis, reactive airway diseases, chronic obstructive pulmonary disease (COPD), pneumonitis, sarcoidosis, hepatitis, nephritis, chronic kidney diseases (CKD), dermatitis, hives, angioedema, psoriasis, optic inflammation, nasal polyps, pemphigus vulgaris, lupus erythematosus, atherosclerosis, nonalcoholic fatty liver disease (NAFLD), nonalcoholic steatohepatitis (NASH), colitis, Crohn's disease, hepatitis, enteritis, polymyositis, leukemia, lymphoma, synovitis, tendonitis, or cerebral edema.

The composition is administered using methods well known in the art; e.g., the composition is administered topically, orally, intravenously, via inhaler or injected directly into the area surrounding inflamed tissue. Because of the unique targeting aspect of the pHLIP® construct, the corticosteroid is specifically targeted to inflamed acidic diseased tissues and delivered into the cytoplasms of inflammatory cells, such as macrophages.

Certain implementations comprise a formulation for a parenteral, a local, or a systemic administration comprising a pHLIP®-linker-CS (where CS is a corticosteroid), as disclosed herein. Formulations comprising a pHLIP®-linker-CS for intravenous, intraarterial, intraperitoneal, intracerebral, intracerebroventricular, intrathecal, intracardiac, intracavernous, intraosseous, intraocular, intravitreal, intramuscular, intradermal, transdermal, transmucosal, intralesional, subcutaneous, topical, epicutaneous, extra-amniotic, intravaginal, intravesical, nasal, or oral administration are presented.

Also provided herein is a formulation comprising a pHLIP®-linker-CS that comprises multiple pHLIP® peptides for systemic administration. In certain embodiments, the formulation is used for the treatment of inflamed and fibrotic tissues.

Provided herein is a method of treating inflamed and fibrotic tissues in a subject, comprising administering to the subject an effective amount of a pH-triggered compound, wherein the compound comprises a corticosteroid.

Also included herein are methods for detecting and/or imaging the targeted delivery to a diseased tissue in a subject comprising administering to the subject a pHLIP®-Linker-CS conjugated with an imaging agent (I.A.), such as I.A.-pHLIP®-Linker-CS. For example, the imaging agent could contain a PET (positron emission tomography) isotope.

Because of the presence of pHLIP® in the composition, the corticosteroid is delivered predominantly to acidic diseased tissue to induce a biological effect predominantly in the targeted tissue. The targeting of a potent corticosteroid preferentially to a diseased tissue compared to a healthy tissue minimizes systemic immunosuppression. In the absence of pHLIP®, prolonged administration of the potent corticosteroid induces systemic immunosuppression, which leads to undesirable and, sometimes, life threatening side effects. Side effects might include osteoporosis, hypertension, diabetes, increased vulnerability to infection, cataracts, glaucoma, thinning of the skin, acne, bruising, myopathy, tachycardia, nausea, insomnia, weight gain, edema, and alterations in mood.

Included herein are pharmaceutical compositions comprising a pH-triggered compound and a pharmaceutically acceptable carrier.

As used herein, “effective” when referring to an amount of a compound refers to the quantity of the compound that is sufficient to yield a desired response without undue adverse side effects commensurate with a reasonable benefit/risk ratio when used in the manner of this disclosure.

In some embodiments, a subject is a mammal. In certain embodiments, the mammal is a rodent (e.g., a mouse or a rat), a primate (e.g., a chimpanzee, a gorilla, a monkey, a gibbon, a baboon), a cow, a camel, a dog, a cat, a horse, a llama, a sheep, or a goat. In preferred embodiments, the subject is a human.

In aspects, provided herein is a method for reducing inflammation in a subject, comprising administering to the subject a composition including a corticosteroid (e.g., dexamethasone) and a pHLIP® peptide. In embodiments, the inflammation includes bleomycin-induced inflammation. In other embodiments, the inflammation is arthritis. In embodiments, the inflammation is dermatitis.

In other aspects, provided herein is a method for enhancing the cytotoxicity of a corticosteroid in a subject, comprising administering to the subject a composition comprising the corticosteroid (e.g., dexamethasone) and a pHLIP® peptide. In embodiments, the corticosteroid and the pHLIP® peptide enhances the cytotoxicity of the corticosteroid by 10%, 20% 30%, 40% 50% 75%, 2-fold, 3-fold, 5-fold, 7-fold, 10-fold or more compared to the level of the corticosteroid alone.

Also within the invention is a method for treating cancer in a subject, comprising administering to the subject a composition comprising (a) a corticosteroid and a pHLIP® peptide, and (b) a chemotherapeutic agent and pHLIP® peptide. For example, the corticosteroid and a pHLIP® peptide composition comprises a pHLIP®-corticosteroid conjugate/construct, e.g., pHLIP®-Dexa. An exemplary chemotherapeutic agent and pHLIP® peptide composition comprises a pHLIP®-chemotherapeutic agent conjugate/construct, e.g., pHLIP-_pHLIP®-calicheamicin. Cancers to be treated in this manner include cancers of the cancers of hematopoetic origin, e.g., cancers of the immune system, immune cells, and/or bone marrow. Such cancer types include leukemias, lymphomas, or myeloma, e.g., multiple myeloma. Leukemia, lymphoma, and multiple myeloma are cancers of the blood-forming organs, e.g., such cancers are hematopoietic neoplasms. In leukemia, the cancerous cells are are generally found circulating in the blood and/or in the bone marrow, while in lymphoma, the cells may aggregate and form masses, or tumors, in lymphatic tissues. Myeloma, e.g., multiple myeloma, is a tumor of the bone marrow. In some examples, the cancer to be treated does not include a discrete solid tumor. The combination therapy (pHLIP®-corticosteroid+pHLIP®-chemotherapeutic agent) is administered intravenously. Alternatively, the combination therapy (pHLIP®-corticosteroid+pHLIP®-chemotherapeutic agent) is administered locally, e.g., directly into the bone marrow such as by direct injection of the combination.

Each embodiment disclosed herein is contemplated as being applicable to each of the other disclosed embodiments. Thus, all combinations of the various elements described herein are within the scope of the invention.

Other features and advantages of the invention will be apparent from the following description of the preferred embodiments thereof, and from the claims. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram of a pHLIP® construct with a corticosteroid (CS) at the membrane-inserting end of the peptide.

FIGS. 2A-C are diagrams of pHLIP® constructs with a CS and a modulator (M) molecule. The modulator is located at the membrane-inserting end of the peptide (FIG. 2A), at the linker location (FIG. 2B), or at the CS location (FIG. 2C), respectively.

FIG. 3 is a diagram of a pHLIP® construct with 2 (or more) corticosteroids (CS) at the membrane-inserting end of the peptide. The corticosteroids can be the same or different.

FIGS. 4A-C are diagrams of pHLIP® constructs with 2 (or more) CS and a M molecule. The modulator is located at the membrane-inserting end of the peptide (FIG. 4A), at the linker location (FIG. 4B), or at the CS cargo location (FIG. 4C), respectively. The corticosteroids can be the same or different.

FIG. 5 is a diagram of a pHLIP construct with a CS at the membrane-inserting end of the peptide and an imaging agent (I) at the membrane non-inserting end.

FIGS. 6A-C are diagrams of pHLIP® constructs with a CS and M (modulator) at the membrane-inserting end of the peptide and an imaging agent (I) at the membrane non-inserting end. The modulator is located at the membrane-inserting end of the peptide (FIG. 6A), at the linker location (FIG. 6B), or at the CS cargo location (FIG. 6C), respectively.

FIG. 7 is a diagram of a pHLIP® construct with multiple CS at the membrane-inserting end of the peptide and an imaging agent at the membrane non-inserting end. CSs can be the same or different.

FIGS. 8A-C are diagrams of a pHLIP® construct with multiple CS and a M (modulator) at the membrane-inserting end of the peptide and an imaging agent at the membrane non-inserting end. The corticosteroids can be the same or different. The modulator is located at the membrane-inserting end of the peptide (FIG. 8A), at the linker location (FIG. 8B), or at the CS cargo location (FIG. 8C), respectively.

FIG. 9 is a diagram of two or more pHLIP® peptides with CS at the membrane-inserting end of the peptide and where the peptides are linked together by a PEG polymer (or any other polymer shown by the purple ribbon). The corticosteroids can be the same or different.

FIGS. 10A-C are diagrams of exemplary pHLIP® constructs with two or more pHLIP® peptides with CS at the membrane-inserting end of the peptide and with the peptides linked together by a PEG polymer (or any other polymer shown by the purple ribbon). The corticosteroids can be the same or different. The modulator is located at the membrane-inserting end of the peptide (FIG. 10A, at the linker location (FIG. 10B), or at the CS cargo location (FIG. 10C), respectively.

FIG. 11 is a diagram of an exemplary pHLIP® constructs with two or more pHLIP® peptides with CS at the membrane-inserting end of the peptide and peptides linked together by a PEG polymer (or any other polymer shown by the purple ribbon). The CSs can be the same or different.

FIGS. 12A-C are diagrams of exemplary pHLIP® constructs with two or more pHLIP® peptides with a CS at the membrane-inserting end of the peptide and the peptides linked together by a PEG polymer (or any other polymer shown by the purple ribbon). The CS can be the same or different. The modulator is located at the membrane-inserting end of the peptide (FIG. 12A), at the linker location (FIG. 12B), or at the CS cargo location (FIG. 12C), respectively.

FIG. 13A is a diagram of an exemplary pHLIP® construct with a CS at the membrane non-inserting end of the peptide. Linker is optional.

FIG. 13B is a diagram of an exemplary pHLIP® construct with a CS and M at the membrane non-inserting end of the peptide, e.g., at the linker location.

FIG. 14A is a diagram of an exemplary pHLIP® construct with two or more CSs at the membrane non-inserting end of the peptide. Linker is optional.

FIG. 14B is a diagram of an exemplary pHLIP® construct with two or more CSs and M at the membrane non-inserting end of the peptide, e.g., at the linker location.

FIG. 15A is a diagram of an exemplary pHLIP® construct with a CS at the membrane non-inserting end of the peptide and two or more peptides linked together.

FIG. 15B is a diagram of an exemplary pHLIP® construct with a CS and M at the membrane non-inserting end of the peptide and two or more peptides linked together. For example, the modulator is present at the linker location.

FIG. 16A is a diagram of an exemplary pHLIP construct with two or more CSs at the membrane non-inserting end of the peptide and two or more peptides linked together.

FIG. 16B is a diagram of an exemplary pHLIP® construct with two or more CSs and M at the membrane non-inserting end of the peptide and two or more peptides linked together. For example, the modulator is present at the linker location.

FIG. 17A are representative images of inflamed lungs obtained from a mouse challenged with lipopolysaccharide (LPS) (Top left—panel “A” and Top right—panel “B”) and control lungs from control mouse (Top right—panel “C” and Bottom right—panel “D”) 21 hrs after single intraperitoneal administration of Indocyanine green (ICG)-pHLIP (0.5 mg/kg, which corresponds to 0.04 mg/kg for human dosage). Images “A” and “C” are photos of lungs and images “B” and “D” are near-infrared fluorescence (NIRF) images of ICG-pHLIP.

FIG. 17B box plot presenting data obtained for each animal (circles), mean (triangle), median (line), and standard error (box itself) values of the NIRF ICG-pHLIP signal from lungs and liver of control and LPS challenged mice 21 hrs after single intraperitoneal administration of ICG-pHLIP (0.5 mg/kg). A statistically significant increase of ICG-pHLIP NIRF in inflamed lungs was observed, while NIRF in liver was indistinguishable between control group and LPS challenged group of animals. P-level was calculated using two-tailed test.

FIG. 18A depicts an image of a chemical structure of dexamethasone-propionyl-PEG(4)-SPDP (Dexa-PEG4) for direct conjugation with pHLIP containing a single Cys residue. For example, the S—S bond in the compound is internal. When it interacts with pHLIP, the S—S bond will be exchanged such that the SH of pHLIP will replace one of sulfurs in this compound.

FIG. 18B depicts an image of a chemical structure of pHLIP-(PEG4)-dexamethasone (pHLIP-Dexa).

FIG. 19 is a schematic presentation of pHLIP-Dexa (FIG. 18B) with cleavable S—S (top arrow) and ester bonds (bottom arrow). These cleavable bonds are cleaved in the cytoplasm to release dexamethasone (Dexa) in its original non-modified form.

FIG. 20 depicts a bar graph showing the concentration of Monocyte chemoattractant protein-1 (MCP-1) in serum of mice treated with LPS and pHLIP-Dexa at doses 1.4 mg/kg (“pH-D 1”), 4.6 mg/kg (“pH-D 2”) and 11.4 mg/kg (“pH-D 3”). All data are mean±SEM. *P<0.05, **P<0.01, significantly different from LPS/Vehicle treated animals. One-way ANOVA with post-hoc t-Test. N=2 in Saline/Vehicle group was performed, N=7-8 in all other groups.

FIG. 21 depicts a bar graph showing the histopathology scores. Inflammation is scored from 0-5 where 0 is normal, 1 is minimal change, 2 is mild change, 3 is moderate change, 4 is marked change, and 5 is severe change. Statistical analysis was performed using a Kruskal-Wallis Test followed by a Dunn's Multiple Comparison Test with Bleomycin+Vehicle set as the control group to which treatments were compared. ****P<0.0001 compared to Bleomycin/Vehicle, **P<0.001 compared to Sham/Vehicle. The inflammation score (scale) is standard for animal assessment of inflammation.

FIG. 22A represents images of haematoxylin and eosin (HE) stained lung tissue from the control group of animals (no inflammation, no treatment).

FIG. 22B represents images of HE stained lung tissue from the group of bleomycin-induced inflammation, and no treatment.

FIG. 22C represents images of HE stained lung tissue from the group of bleomycin-induced inflammation treated with pHLIP-Dexa.

FIG. 22D represents images of HE stained lung tissue from the group of bleomycin-induced inflammation treated with Dexa.

FIG. 23 depicts a graph showing the total clinical score for arthritis (maximum 20) recorded daily, 5 times a week, beginning Day 28 after the induction of pHLIP-DEXA, Dexamethasone at 0.46 mg/kg or Dexamethasone at 20 mg/kg). Statistical analysis was performed using a two-way ANOVA with post-hoc Fisher's uncorrected LSD test. There was a significant effect of treatment (P<0.0001): *P<0.05, **P<0.01, ***P<0.001, ****P<0.0001, ns—not significant. The inflammation score (scale) is standard for animal assessment of inflammation.

FIG. 24 depicts a graph showing an average hind paw clinical score for arthritis (maximum 4) recorded daily, 5 times a week, beginning Day 28 after the induction. Statistical analysis was performed using a two-way ANOVA with post-hoc Fisher's uncorrected LSD test. There was a significant effect of treatment (P<0.0001): *P<0.05, **P<0.01, ***P<0.001, ****P<0.0001, ns—not significant.

FIG. 25 depicts a graph showing the body weight of animals, which was recorded three times a week beginning Day 28 after the induction.

FIG. 26 depicts a bar graph showing dermatitis severity which was measured on a scale 0 to 3 (0—no evidence of erythema or swelling; 1—mild swelling; 2—mild erythema and moderate selling; 3—erythema and severe swelling), 24 hrs after a challenge with oxazolone. Data are mean±SEM and analyzed by One-way ANOVA as applicable (**P<0.01, ***P<0.001, ****P<0.00001, compared to Sham/Vehicle, n=8/group).

FIG. 27 depicts a bar graph showing ear thickness that was measured 24 hrs after a challenge with oxazolone and expressed as a percentage of change from the baseline. Data are mean±SEM and analyzed by One-way ANOVA as applicable (****P<0.00001, compared to Sham/Vehicle, n=8/group).

FIG. 28 depicts an image of a chemical structure of calicheamicin modified with succinimidyl 3-(2-pyridyldithio)propionate (SPDP, outlined in box) for direct conjugation with pHLIP containing a single Cys residue. The S—S bond within the outlined box is the point of conjugation with pHLIP.

FIG. 29 depicts a bar graph showing myeloma cancer cells (RPMI 8226 myeloma cells) viability after treatment with pHLIP-calicheamicin in absence and presence of pHLIP-Dexa.

### DETAILED DESCRIPTION

Local acidification develops during acute and chronic inflammation as a result of the infiltration and activation of inflammatory cells (mostly macrophages) in the tissue, which leads to increased energy and oxygen demand, accelerated glucose consumption via glycolysis (especially in the case of ml macrophages) and thus increased lactic acid secretion. Examples of local acidosis in inflammation include atherosclerotic lesions, airways of patients suffering from chronic inflammatory obstructive lung disease, kidney and liver inflammation, and the joint fluids in patients with arthritis. For example, in freshly removed human carotid atherosclerotic plaques, the averaged pH values are about 6.8. In acute exacerbations of asthma, the exhaled breath condensate had an average pH of 5.2 compared with pH 7.7 in healthy subjects, and anti-inflammatory corticosteroid therapy normalized the airway pH. Synovial fluid pH in rheumatic joints may reach values of 6.8-7.1, whereas the pH of normal synovial fluid ranges from 7.4 to 7.8.

A pH Low Insertion Peptide (pHLIP®) is a water-soluble membrane peptide that can sense pH, especially the pH at the surfaces of inflammatory cells, where the pH is at its lowest. pHLIP® interacts weakly with a cell membrane at neutral pH, without insertion into the lipid bilayer; however, at slightly acidic pH (<7.0), pHLIP® inserts into the cell membrane and forms a stable transmembrane helix. pHLIP® targets acidity at the surfaces of macrophages within tumors, atherosclerotic plaques and sites of inflammatory arthritis, kidney, lungs or skin. By binding a pHLIP®, or pHLIP® equivalent, to a corticosteroid, it is possible to specifically deliver the corticosteroid directly to acidic inflamed diseased tissues to induce local (but not systemic) immunosuppression and, thus, locally suppress inflammatory processes.

Delivering potent corticosteroids using pHLIP® peptides therefore allows selective targeting of diseased tissue (e.g. sites of inflammation) to increase the efficacy of corticosteroid treatment. A significant advantage of this approach is that the targeted delivery of corticosteroids mediated by the pHLIP® constructs described herein is associated with the reduction of systemic immunosuppression, which is the main problem in prolonged use of potent corticosteroids. As a result, the use of the pHLIP® constructs described herein allow the longer duration of administration of effective corticosteroids while reducing serious side effects.

Another advantage of pHLIP-conjugated drugs (e.g., corticosteroids and/or chemotherapeutic agents such as cancer drugs) is that the pHLIP®-conjugate drugs remain in the area of inflammation (due to the low pH) for longer periods of time compared to administration of the drug in the absence of pHLIP®. The pHLIP® mediates delivery of drug into cells with low pH thereby retaining the drug at the site of inflammation to be treated. In contrast, drugs, e.g., small molecule drugs, disseminate rapidly from such a site. For example, retention at a site of local administration, e.g., of an inflamed joint such as in the case of arthritis or in the case of delivery to bone marrow, is a significant advantage over administration of the drug alone.

The invention provides compounds comprising peptides having the properties of preferential affinity for and insertion across membrane lipid bilayers at low pH, together with corticosteroids to promote a biological immunosuppressive effect in targeted acidic diseased tissues. Corticosteroids alone cannot distinguish between diseased and healthy tissue, thus affecting both, which leads to systemic immunosuppression and serious side effects and prevents the prolonged administration of corticosteroids. pHLIP® peptides mediate the direction/delivery/targeting of corticosteroids to diseased tissues, representing a significant clinical advantage over conventional corticosteroid formulations.

The invention uses pHLIP® to target inflamed and fibrotic tissues to specifically deliver corticosteroids into cells (such as macrophages) to promote immunosuppression predominantly within a targeted tissue. There are 3 major aspects of the invention: i) targeting of corticosteroids to acidic diseased tissues to induce immunosuppression predominantly within the diseased tissue; ii) sparing of normal healthy organs and tissue to reduce side effects associated with prolonged uses of corticosteroids; iii) direct delivery of corticosteroids into the cytoplasms of cells in diseased tissues, avoiding endosomal uptake, which requires endosomal escape for therapy. Thus, pHLIP® guides or targets corticosteroids for effective intracellular delivery within acidic diseased tissues.

### Corticosteroids

Corticosteroids are drugs closely related to cortisol, a hormone which is naturally produced in the adrenal cortex. Corticosteroids or glucocorticoids were considered to be miraculous, since they demonstrated an excellent therapeutic effect in 1948 on a group of arthritis patients. Since that time, corticosteroids have been used for treatment of variety of diseases states. However, as the use of corticosteroids expanded over the years, side effects emerged. Side effects depend on the dose, route of administration and duration of corticosteroids administration. Short-term use can cause weight gain, puffy face, nausea, mood swings, and trouble sleeping, thinner skin, acne, unusual hair growth, and spikes in blood sugar and blood pressure. Because corticosteroids turn down or reduce the activity of the immune system, taking them makes a subject/patient being treated with corticosteroids more likely to get infections. Long-term use of corticosteroids can cause serious side effects like osteoporosis, slow growth in children, and a life-threatening condition called adrenal insufficiency, in which the body cannot respond to stress such as surgery or illnesses, muscle weakness, eye problems (including cataracts), and a higher risk of diabetes. Cushing's syndrome (a condition characterized by a combination of various symptoms described above) appears in a result of prolonged use of corticosteroids. Corticosteroids are powerful drugs that are valuable if they can be targeted to diseased tissues and induce anti-inflammatory effects locally at the site of a diseased/inflamed/acidic tissue while leaving non-diseased tissues untreated, e.g., minimally or unaffected by the corticosteroid. The non-diseased tissue is therefore not subject to the adverse side effects that occur with corticosteroids delivered in the absence of the association or tether to a pHLIP® peptide.

pHLIP® Constructs

The invention provides compositions and methods to target acidic diseased tissues with pHLIP® to specifically deliver potent corticosteroids to the inflamed tissue, bypass endocytotic uptake and deliver corticosteroids in cytoplasm of cells (macrophages) in targeted diseased tissue, and promote biological effects specifically within the targeted tissue only (or predominantly). As described above, it would be very advantageous to target potent corticosteroids to diseased tissue to reduce side effects. The constructs described herein mediate such specific or preferential targeting.

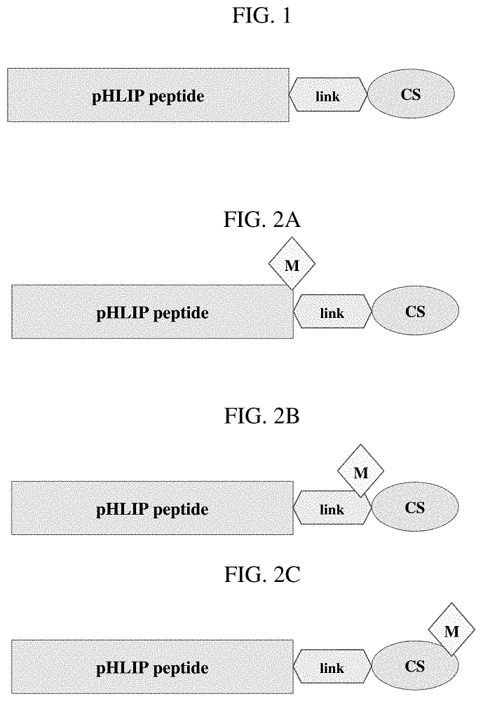

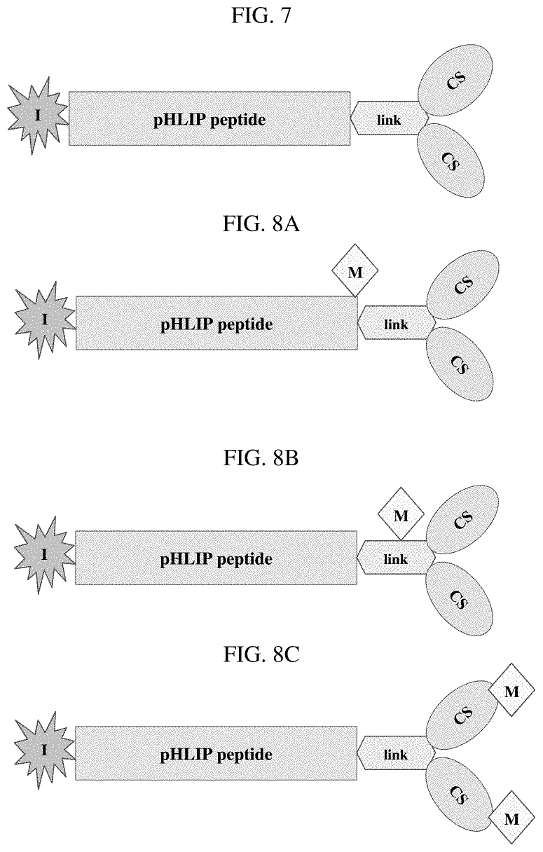

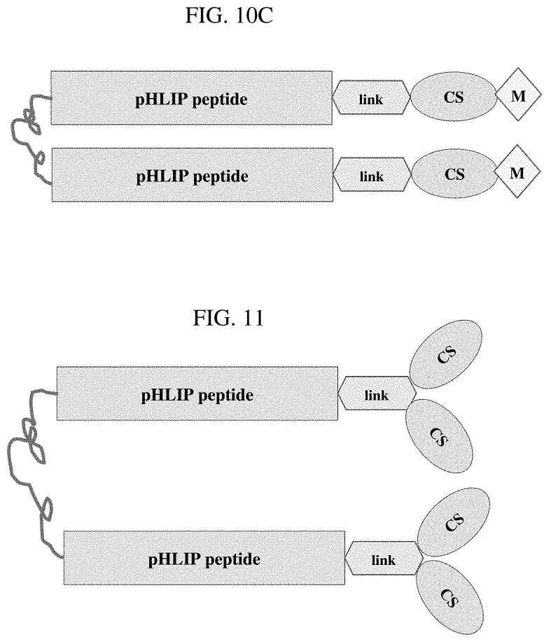

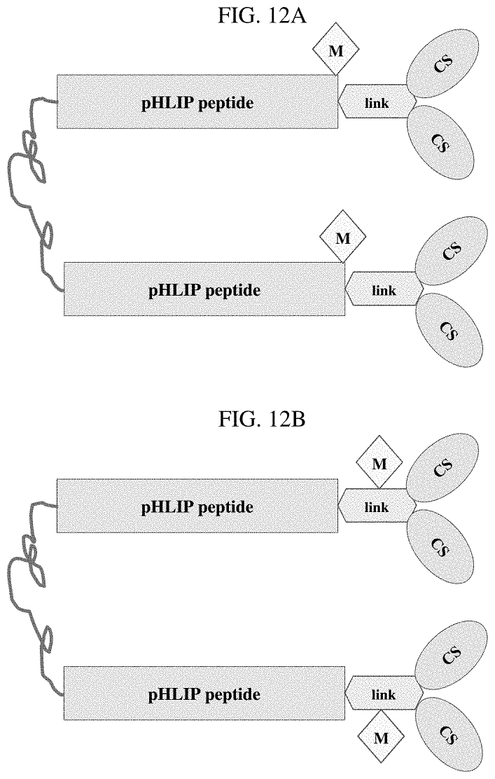

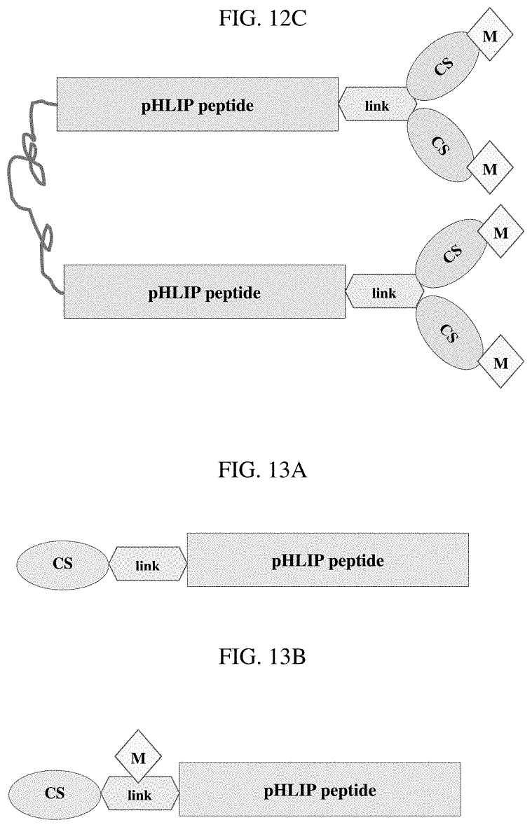

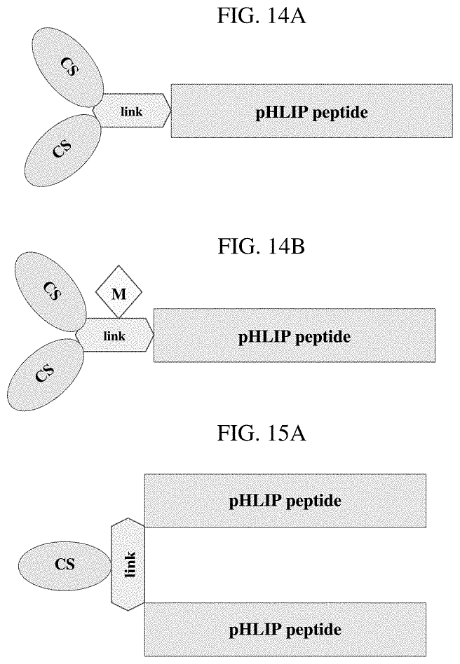

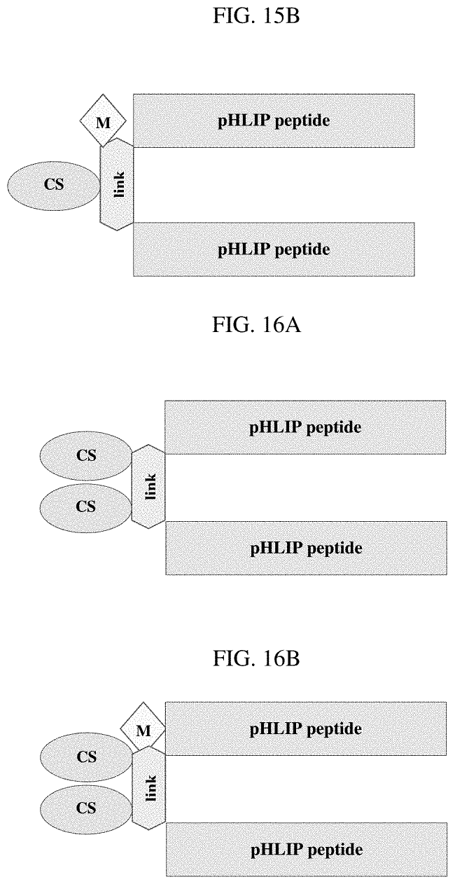

General representations of pHLIP® compounds comprising pHLIP® peptide and a corticosteroids are shown in FIGS. 1-16 and described below.

FIG. 1 shows a corticosteroid (CS) molecule linked to the membrane-inserting end of a pHLIP® peptide (and an optional linker).

FIGS. 2A-C show one or more polar modulator molecules (M) are optionally attached to enhance targeting or solubility.

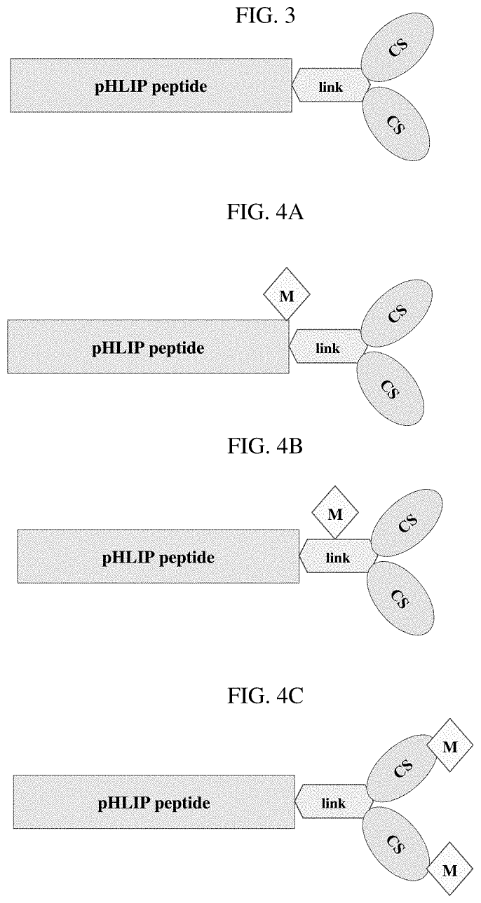

FIG. 3 shows multiple CS molecules linked to a single pHLIP® peptide at the peptide membrane-insertion end.

FIGS. 4A-C show multiple CS molecules linked to a single pHLIP® peptide with one or more modulator molecules at the peptide membrane-insertion end.

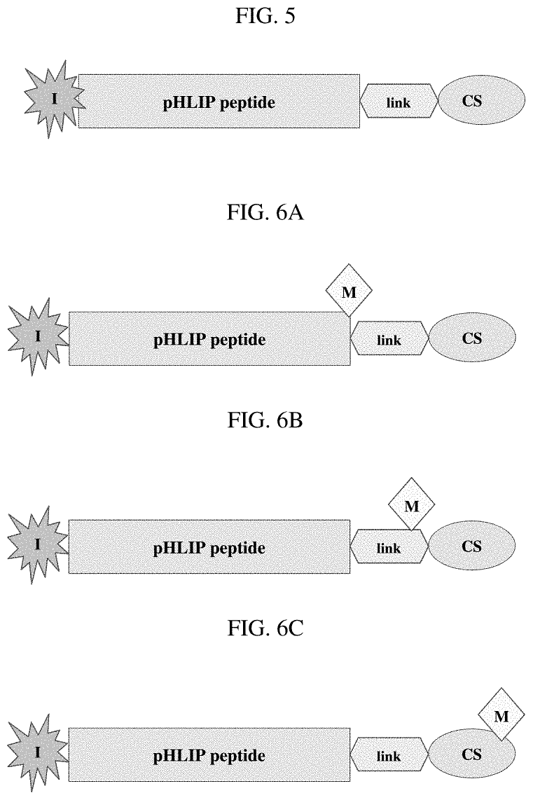

FIGS. 5, 6A-C, 7, and 8A-C depict pHLIP® compounds that can carry one or more imaging agents (I) (or other molecules) at their pHLIP® peptide membrane-non-inserting ends.

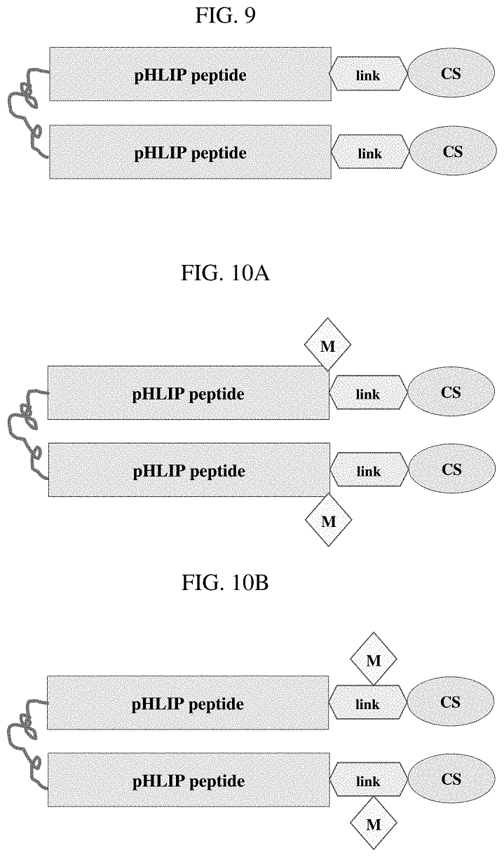

FIG. 9 shows two or more pHLIP® peptides with a CS linked together via linker molecule.

FIGS. 13A-B, 14A-B, 15, A-B, and 16A-B show pHLIP® peptides with a CS at their non-membrane-insertion ends.

Exemplary constructs include a Var3 pHLIP® sequence ADDQNPWRAYLDLLFPTDTLLLDLLWCA (SEQ ID NO: 3) or variations thereof, e.g., sequences provided in Tables below and in references cited herein (and incorporated by reference).

CS(s) is linked to pHLIP® peptide(s) via a cleavable or non-cleavable link(s). For example, the cleavable link can be a disulfide bond, or acid-liable, or ester bond link. In other examples, the cleavable link is a self-immolating link.

### Dexamethasone and Betamethasone

Dexamethasone and Betamethasone belong to the group of potent corticosteroids, which suppress pro-inflammatory M1 macrophages and reduce inflammatory signals. They demonstrate a profound anti-inflammatory properties that are attributed to several different molecular mechanisms of action that involve inhibition of several synthesis pathways, which include: i) phospholipase-A2 biochemical activity inhibition resulting in a diminished arachidonic acid substrate availability for prostaglandin and leukotriene synthesis; ii) resulting in a reduced production of tumor necrosis factor-α and Th1 interleukins; iii) reduced IL-5 production; and iv) suppression of IFN-γ-induced major histocompatibility antigen Type II expression accompanied by an induced synthesis of endogenous IL-10 that potently exerts profound anti-inflammatory properties. Influences of these corticosteroids on immune cellular function are in part related to their i) prevention or reduction of leukocyte degranulation; ii) inhibition of macrophage phagocytosis; and iii) promotion of overt lymphocyte cytolysis.

pHLIP® Peptides

pHLIP® peptides are a family of peptides that (1) target acidic tissues in vivo, including tumors, and (2) can deliver polar molecules into cells, releasing them in the cytoplasm. The peptides are soluble as mostly unstructured monomers in aqueous solution, bind as unstructured monomers to the surfaces of bilayers or membranes, and fold to make helices that insert across membranes when the environment is acidic. Cargo molecules can be delivered into cells characterized by an acidic surface/microenvironment by the presence of the cargo molecule on the inserting end of a pHLIP® peptide. A water-soluble therapeutic molecule can be attached as cargo to the inserting end of pHLIP by a bond that is unstable inside a cell, but stable outside the cell. When a pHLIP® peptide folds at low pH and delivers the cargo across the membrane, the bond breaks and the cargo is released in the cytosol, where it has a therapeutic effect.

An example of a wild type (WT) is AEQNPIYWARYADWLFTTPLLLLDLALLVDADEGT (SEQ ID NO: 4) in which AEQNPIY (SEQ ID NO: 5) represents a flanking sequence, WARYADWLFTTPLLLLDLALLV (SEQ ID NO: 6) represents a membrane-inserting sequence, and DADEGT (SEQ ID NO: 7) represents a flanking sequence.

Other exemplary pHLIP® peptides are shown in the Tables below.

TABLE 1
Exemplary pHLIP® peptides

| Name | Sequence | SEQ ID No. |
|---|---|---|
| WT-1 | GGEQNPIYWARYADWLFTTPLLLLDLALLVDADEGT | SEQ. ID NO. 8 |
| WT-2 | AEQNPIYWARYADWLFTTPLLLLDLALLVDADEGT | SEQ. ID NO. 9 |
| Var3-CK | ADDQNPWRAYLDLLFPTDTLLLDLLWCKA | SEQ. ID NO. 10 |
| Var3-WT-Cys | ADDQNPWRAYLDLLFPTDTLLLDLLWDADECG | SEQ. ID NO. 11 |
| Var3-WT-Lys | ADDQNPWRAYLDLLFPTDTLLLDLLWDADEKG | SEQ. ID NO. 12 |
| Var3-WT-KC | ADDQNPWRAYLDLLFPTDTLLLDLLWDADEKCG | SEQ. ID NO. 13 |
| Cys-Var3-WT | ACDDQNPWRAYLDLLFPTDTLLLDLLWDADEG | SEQ. ID NO. 14 |
| Lys-Var3-WT | AKDDQNPWRAYLDLLFPTDTLLLDLLWDADEG | SEQ. ID NO. 15 |
| WT-Cys1 | AAEQNPIYWARYADWLFTTPLLLLDLALLVDADEGTCG | SEQ. ID NO. 16 |
| WT-Cys2 | Ac-AEQNPIYWARYADWLFTTPLLLLDLALLVDADEGCT | SEQ ID NO: 17 |
| WT-Cys3 | GGEQNPIYWARYADWLFTTPLLLLDLALLVDADEGTCG | SEQ. ID NO. 18 |
| Cys-WT1 | Ac-ACEQNPIYWARYADWLFTTPLLLLDLALLVDADEGTG | SEQ. ID NO. 19 |
| Var0-NT | ACEQNPIYWARYADWLFTTPLLLLDLALLVDADEGT | SEQ. ID NO. 20 |
| Lys-WT1 | AKEQNPIYWARYADWLFTTPLLLLDLALLVDADEGT | SEQ. ID NO. 21 |
| Lys-WT2 | Ac-AKEQNPIYWARYADWLFTTPLLLLDLALLVDADEGTG | SEQ ID NO: 22_ |
| WT-KC | AAEQNPIYWARYADWLFTTPLLLLDLALLVDADEGTKCG | SEQ. ID NO. 23 |
| K-WT-C | AKEQNPIYWARYADWLFTTPLLLLDLALLVDADECT | SEQ. ID NO. 24 |
| N-pHLIP | ACEQNPIYWARYANWLFTTPLLLLNLALLVDADEGTG | SEQ. ID NO. 25 |
| N-pHLIP-b | ACEQNPIYWARYANVVLETTPLLLLNLALLVDADEGT | SEQ ID NO: 26 |
| K-pHLIP | ACEQNPIYWARYAKWLFTTPLLLLKLALLVDADEGTG | SEQ. ID NO. 27 |
| NNQ | GGEQNPIYWARYADWLFTTPLLLLDLALLVNANQGT | SEQ. ID NO. 28 |
| D25A | AAEQNPIYWARYADWLFTTPLLLLALALLVDADEGT | SEQ. ID NO. 29 |
| D25A-KC | Ac-AAEQNPIYWARYADWLFTTPLLLLELALLVDADEGTKCG | SEQ ID NO: 30_ |
| D14A | AAEQNPIYWARYAAWLFTTPLLLLDLALLVDADEGT | SEQ. ID NO. 31 |
| P20A | AAEQNPIYWARYADWLFTTALLLLDLALLVDADEGT | SEQ. ID NO. 32 |
| D25E | AAEQNPIYWARYADWLFTTPLLLLELALLVDADEGT | SEQ. ID NO. 33 |
| D14E | AAEQNPIYWARYAEWLFTTPLLLLDLALLVDADEGT | SEQ. ID NO. 34 |
| 3D | AAEQNPIIYWARYADWLFTDLPLLLLDLLALLVDADEGT | SEQ. ID NO. 35 |
| R11Q | GEQNPIYWAQYADWLFTTPLLLLDLALLVDADEGTCG | SEQ. ID NO. 36 |
| D25Up | GGEQNPIYWARYADWLFTTPLLLDLLALLVDADEGTCG | SEQ. ID NO. 37 |
| D25Down | GGEQNPIYWARYADWLFTTPLLLLLDALLVDADEGTCG | SEQ. ID NO. 38 |
| D14Up | GGEQNPIYWARYDAWLFTTPLLLLDLALLVDADEGTCG | SEQ. ID NO. 39 |
| D14Down | GGEQNPIYWARYAWDLFTTPLLLLDLALLVDADEGTCG | SEQ. ID NO. 40 |
| P20G | AAEQNPIYWARYADWLFTTGLLLLDLALLVDADEGT | SEQ. ID NO. 41 |
| H1-Cys | DDDEDNPIYWARYADWLFTTPLLLLHGALLVDADECT | SEQ. ID NO. 42 |
| H1 | DDDEDNPIYWARYADWLFTTPLLLLHGALLVDADET | SEQ ID NO: 43_ |
| H2-Cys | DDDEDNPIYWARYAHWLFTTPLLLLHGALLVDADEGCT | SEQ. ID NO. 44 |
| Cys-H2 | CDDDEDNPIYWARYAHWLFTTPLLLLHGALLVDADET | SEQ ID NO: 45_ |
| H2 | DDDEDNPIYWARYAHWLFTTPLLLLHGALLVDADEGT | SEQ ID NO: 46 |
| H2N-Cys | DDDEDNPIYWARYAHWLFTTPLLLLHGALLVNADECT | SEQ. ID NO. 47 |
| H2N | DDDEDNPIYWARYAHWLFTTPLLLLHGALLVNADEGT | SEQ ID NO: 48_ |
| H2N2-Cys | DDDEDNPIYWARYAHWLFTTPLLLLHGALLVNANECT | SEQ. ID NO. 49 |
| H2N2 | DDDEDNPIYWARYAHWLFTTPLLLLHGALLVNANEGT | SEQ ID NO: 50_ |
| 1a-Trp | AEQNPIYWARYADFLFTTPLLLLDLALLVDADET | SEQ. ID NO. 51 |
| 1b-Trp | AEQNPIYFARYADWLETTPLLLLDLALLVDADEGT | SEQ. ID NO. 52 |
| 1c-Trp | AEQNPIYFARYADFLFTTPLLLLDLALLWDADET | SEQ. ID NO. 53 |
| Fast-1 or Var1 | AKEDQNPYWARYADWLFTTPLLLLDLALLVDG | SEQ. ID NO. 54 |
| Var1-2D1D | ACEDQNPYWARYADWLFTTPLLLLDLALLVDG | SEQ. ID NO. 55 |
| Fast1-Cys or | AEDQNPYWARYADWLFTTPLLLLDLALLVDCG | SEQ. ID NO. 56 |
| Var1-2D1D-Cys | | |
| Fast1-E-Cys or | AEDQNPYWARYADWLFTTPLLLLELALLVECG | SEQ. ID NO. 57 |
| Var1E | AKEDQNDPYWARYADWLFTTPLLLLDLALLVG | SEQ ID NO: 58_ |
| Fast1-E-Lys | AKEDQNPYWRAYADLFTPLTLLDLLALWDG | SEQ. ID NO. 59 |
| Fast2 or Var2 | | |
| Fast2-E-Cys or | AEDQNPYWARYADWLFTTPLLLLELALLVCG | SEQ ID NO: 60_ |
| Var2E | | |
| Var2-2D1D | ACEDQNPYWRAYADLFTPLTLLDLLALWDG | SEQ. ID NO. 61 |
| Var3-3D | ACDDQNPWRAYLDLLFPTDTLLLDLLW | SEQ. ID NO. 62 |
| Var3-3D-cys | AKDDQNPWRAYLDLLFPTDTLLLDLLWC | SEQ ID NO: 63_ |
| Var4-3E | ACEEQNPWRAYLELLFPTETLLLELLW | SEQ ID NO: 64 |
| Var5-3Da | ACDDQNPWARYLDWLFPTDTLLLDL | SEQ ID NO: 65 |
| Var6-3Db | CDNNNPWRAYLDLLFPTDTLLLDW | SEQ ID NO: 66 |
| Var8-3Eb | CEEQQPWAQYLELLFPTETLLLEW | SEQ ID NO: 67 |
| Var9-3Ec | CEEQQPWRAYLELLFPTETLLLEW | SEQ ID NO: 68 |
| Var15-2N | CDDDDDNPNYWARYANWLFTTPLLLLNGALLVEAEET | SEQ ID NO: 69 |
| Var16-2P | CDDDDDNPNYWARYAPWLFTTPLLLLPGALLVEAEE | SEQ ID NO: 70_ |

TABLE 2
Exemplary pHLIP® peptides

| Name | Sequence | SEQ ID NO. |
|---|---|---|
| Var14-Rev | Ac-TEDADVLLALDLLLLPTTFL | SEQ. ID NO. 71 |
| | WDAYRAWYPNQECA-Am | |
| Sh | AEQNPIYWARYADWLFTTPL | SEQ. ID NO. 72 |
| Sh-Cys | AEQNPIYWARYADWLFTTPCL | SEQ. ID NO. 73 |
| Cys-Sh | ACEQNPIYWARYADWLFTTPL | SEQ. ID NO. 74 |
| Sh-1Trp | AEQNPIYFARYADWLETTPL | SEQ. ID NO. 75 |
| Sh-W2 | AEQNPIYFARYADLLEPTTLAW | SEQ ID NO: 76 |
| Sh-W1 | AEQNPIYWARYADLLFPTTLAF | SEQ ID NO: 77 |
| Sh-2W | AEQNPIYWARYADLLFPTTLAW | SEQ ID NO: 78 |
| Sh-1D | KEDQNPWARYADLLFPTTLAW | SEQ. ID NO. 79 |
| Sh-1Db | KEDQNPWARYADLLFPTTLW | SEQ ID NO: 80 |
| Var12-1D | ACEDQNPWARYADLLFPTTLAW | SEQ. ID NO. 81 |
| Var10-2D | ACEDQNPWARYADWLFPTTLLLLD | SEQ. ID NO. 82 |
| Var13-1E | ACEEQNPWARYAELLFPTTLAW | SEQ. ID NO. 83 |
| Var11-2E | ACEEQNPWARYAEWLFPTTLLLLE | SEQ. ID NO. 84 |
| Var7-3E | ACEEQNPWARYLEWLFPTETLLLEL | SEQ. ID NO. 85 |
| Var7-3Eb | ACEEQNPQAEYAEWLFPTTLLLLE | SEQ ID NO: 86 |

“Ac” means Acetylated N-terminus
“Am” means Amidated C-terminus

TABLE 3
Coded and exemplary non-coded amino acids including
L-isomers, D- isomers, alpha-isomers, beta-isomers,
glycol-, and methyl- modifications.

| No. | Abbrev | Name |
|---|---|---|
| 1 | Ala | Alanine |
| 2 | Arg | Arginine |
| 3 | Asn | Asparagine |
| 4 | Asp | Aspartic acid |
| 5 | Cys | Cysteine |
| 6 | Gln | Glutamine |
| 7 | Glu | Glutamic acid |
| 8 | Gly | Glycine |
| 9 | His | Histidine |
| 10 | Ile | Isoleucine |
| 11 | Leu | Leucine |
| 12 | Lys | Lysine |
| 13 | Met | Methionine |
| 14 | Phe | Phenylalanine |
| 15 | Pro | Proline |
| 16 | Ser | Serine |
| 17 | Thr | Threonine |
| 18 | Trp | Tryptophan |
| 19 | Tyr | Tyrosine |
| 20 | Val | Valine |
| 21 | Sec | Selenocysteine |
| 22 | Sem | Selenomethionine |
| 23 | Pyl | Pyrrolysine |
| 24 | Aad | Alpha-aminoadipic acid |
| 25 | Acpa | Amino-caprylic acid |
| 26 | Aecys | Aminoethyl cysteine |
| 27 | Afa | Aminophenyl acetate |
| 28 | Gaba | Gamma-aminobutyric acid |
| 29 | Aiba | Aminoisobutyric acid |
| 30 | Aile | Alloisoleucine |
| 31 | AIg | Allylglycine |
| 32 | Aba | Amino-butyric acid |
| 33 | Aphe | Amino-phenylalanine |
| 34 | Brphe | Bromo-phenylalanine |
| 35 | Cha | Cyclo-hexylalanine |
| 36 | Cit | Citrulline |
| 37 | Clala | Chloroalanine |
| 38 | Cie | Cycloleucine |
| 39 | Clphe | Fenclonine (or chlorophenylalanine) |
| 40 | Cya | Cysteic acid |
| 41 | Dab | Diaminobutyric acid |
| 42 | Dap | Diaminopropionic acid |
| 43 | Dap | Diaminopimelic acid |
| 44 | Dhp | Dehydro-proline |
| 45 | Dhphe | DOPA (or 3,4- |
| 46 | Fphe | dihydroxyphenylalanine) |
| 47 | Gaa | Fluorophenylalanine |
| 48 | Gla | Glucosaminic acid |
| 49 | Hag | Gamma-carboxyglutamic acid |
| 50 | Hlys | Homoarginine |
| 51 | Hnvl | Hydroxylysine |
| 52 | Hog | Hydroxynorvaline |
| 53 | Hoph | Homoglutamine |
| 54 | Has | Homophenylalanine |
| 55 | Hse | Homoserine |
| 56 | Hpr | Homocysteine |
| 57 | Iphe | Hydroxyproline |
| 58 | Ise | Iodo-phenylalanine |
| 59 | Mle | Isoserine |
| 60 | Msmet | Methyl-leucine |
| 61 | Nala | Methionine-methylsulfonium chloride |
| 62 | Nle | Naphthyl-alanine |
| 63 | Nmala | Norleucine (or 2-aminohexanoic acid) |
| 64 | Nva | N-methyl-alanine |
| 65 | Obser | Norvaline (or 2-aminopentanoic acid) |
| 66 | Obtyr | O-benzyl-serine |
| 67 | Oetyr | O-benzyl-tyrosine |
| 68 | Omser | O-ethyl-tyrosine |
| 69 | Omthr | O-methyl-serine |
| 70 | Omtyr | O-methy-threonine |
| 71 | Orn | O-methyl-tyrosine |
| 72 | Pen | Ornithine |
| 73 | Pga | Penicillamine |
| 74 | Pip | Pyroglutamic acid |
| 75 | Sar | Pipecolic acid |
| 76 | Tfa | Sarcosine |
| 77 | Thphe | Trifluoro-alanine |
| 78 | Vig | Hydroxy-Dopa |
| 79 | Aaspa | Vinylglycine |
| 80 | Ahdna | Amino-aminoethylsulfanylpropanoic |
| 81 | Ahoha | acid |
| 82 | Ahsopa | Amino-hydroxy-dioxanonanolic acid |
| 83 | Tyr(Me) | Amino-hydroxy-oxahexanoic acid |
| 84 | MTrp | Amino- |
| 85 | pTyr | hydroxyethylsulfanylpropanoic acid |
| 86 | pSer | Methoxyphenyl-methylpropanyl |
| 87 | pThr | oxycarbonylamino propanoic acid |
| 88 | BLys | Methyl-tryptophan |
| 89 | Hyp | Phosphorylated Tyr |
| 90 | Phg | Phosphorylated Ser |
| 91 | Cha | Phosphorylated Thr |
| 92 | Chg | BiotinLys |
| 93 | Nal | Hydroproline |
| 94 | Pal | Phenylglycine |
| 95 | Pra | Cyclohexyl-alanine |
| 96 | Gly(allyl) | Cyclohexylglycine |
| 97 | Pen | Naphthylalanine |
| 98 | MetO | Pyridyl-alanine |
| 99 | Pea | Propargylglycine |
| 100 | Ac-Lys | Pentenoic acid |
| | | Penicillamine |
| | | Methionine sulfoxide |
| | | Pyroglutamic acid |
| | | Acetylation of Lys |

TABLE 4
Non-limiting examples of protonatable residues
and their substitutions including L- isomers,
D- isomers, alpha-isomers, and beta-isomers.

| Original Residue | Exemplary amino acids substitution |
|---|---|
| Asp (D) | Glu (E); Gla (Gla); Aad (Aad) |
| Glu (E) | Asp (D); Gla (Gla); Aad (Aad) |

TABLE 5
Examples of coded amino acid substitutions

| Original Residue | Substitution |
|---|---|
| Ala (A) | Gly; Ile; Leu; Met; Phe; Pro; Trp; Tyr; Val |
| Arg (R) | Lys |
| Asn (N) | Gln; His |
| Asp (D) | Glu |
| Cys (C) | Ser; Met |
| Gln (Q) | Asn; His |
| Glu (E) | Asp |
| Gly (G) | Ala; Ile; Leu; Met; Phe; Pro; Trp; Tyr; Val |
| His (H) | Asn; Gln |
| Ile (I) | Ala; Gly; Leu; Met; Phe; Pro; Trp; Tyr; Val |
| Leu (L) | Ala; Gly; Ile; Met; Phe; Pro; Trp; Tyr; Val |
| Lys (K) | Arg |
| Met (M) | Ala; Gly; Leu; Ile; Phe; Pro; Trp; Tyr; Val |
| Phe (F) | Ala; Gly; Leu; Ile; Met; Pro; Trp; Tyr; Val |
| Pro (P) | Ala; Gly; Leu; Ile; Met; Phe; Trp; Tyr; Val |
| Ser (S) | Thr |
| Thr (T) | Ser |
| Trp (W) | Ala; Gly; Leu; Ile; Met; Pro; Phe; Tyr; Val |
| Tyr (Y) | Ala; Gly; Leu; Ile; Met; Pro; Phe; Trp; Val |
| Val (V) | Ala; Gly; Leu; Ile; Met; Pro; Phe; Trp; Tyr |

TABLE 6
Non-limiting examples of membrane-inserting
sequences belonging to different groups of
pHLIP® peptides. Each protonatable residue
(shown in bold) could be replaced by
its substitution from Table 4. Each non-polar
residue could be replaced by its coded amino
acid substitution from Table 5, and/or non-coded
amino acid substitutions from Table 3.
Bolded residues are protanable residues.

| Groups | Sequences | SEQ ID NO: |
|---|---|---|
| WT-BRC | WARYADWLFTTPLLLLDLALL | SEQ ID NO: 87 |
| | YARYADWLFTTPLLLLDLALL | SEQ ID NO: 88 |
| | WARYSDWLFTTPLLLYDLGLL | SEQ ID NO: 89 |
| | WARYTDWFTTPLLLYDLALLA | SEQ ID NO: 90 |
| | WARYTDWLFTTPLLLYDLGLL | SEQ ID NO: 91 |
| | WARYADWLFTTPLLLLDLSLL | SEQ ID NO: 92 |
| WT-BRC | LLALDLLLLPTTFLWDAYRAW | SEQ ID NO: 93 |
| Reverse | LLALDLLLLPTTFLWDAYRAY | SEQ ID NO: 94 |
| | LLGLDYLLLPTTFLWDSYRAW | SEQ ID NO: 95 |
| | ALLALDYLLLPTTFWDTYRAW | SEQ ID NO: 96 |
| | LLGLDYLLLPTTFLWDTYRAW | SEQ ID NO: 97 |
| | LLSLDLLLLPTTFLWDAYRAW | SEQ ID NO: 98 |
| ATRAM | GLAGLLGLEGLLGLPLGLLEGLWLGL | SEQ ID NO: 99 |
| ATRAM | LGLWLGELLGLPLGLLGELGLLGALG | SEQ ID NO: 100 |
| Reverse | | |
| Var3 | WRAYLDLLFPTDTLLLDLLW | SEQ ID NO: 101 |
| Var3 | WLLDLLLTDTPFLLDLYARW | SEQ ID NO: 102 |
| Reverse | | |
| Var7 | WARYLEWLFPTETLLLEL | SEQ ID NO: 103 |
| | WAQYLELLFPTETLLLEW | SEQ ID NO: 104 |
| Var7 | LELLLTETPFLWELYRAW | SEQ ID NO: 105 |
| Reverse | WELLLTETPFLLELYQAW | SEQ ID NO: 106 |
| Single | WLFTTPLLLLNGALLVE | SEQ ID NO: 107 |
| D/E | WLFTTPLLLLPGALLVE | SEQ ID NO: 108 |
| | WARYADLLFPTTLAW | SEQ ID NO: 109 |
| Single | EVLLAGNLLLLPTTFLW | SEQ ID NO: 110 |
| D/E | EVLLAGPLLLLPTTFLW | SEQ ID NO: 111 |
| Reverse | WALTTPFLLDAYRAW | SEQ ID NO: 112 |
| pHLIP®- | NLEGFFATLGGEIALWSLVVLAIE | SEQ ID NO: 113 |
| Rho | EGFFATLGGEIALWSDVVLAIE | SEQ ID NO: 114 |
| | EGFFATLGGEIPLWSDVVLAIE | SEQ ID NO: 115 |
| pHLIP®- | EIALVVLSWLAIEGGLTAFFGELN | SEQ ID NO: 116 |
| Rho | EIALVVDSWLAIEGGLTAFFGE | SEQ ID NO: 117 |
| Reverse | EIALVVDSWLPIEGGLTAFFGE | SEQ ID NO: 118 |
| pHLIP®- | ILDLVFGLLFAVTSVDFLVQW | SEQ ID NO: 119 |
| CA9 | | |
| pHLIP®- | WQVLFDVSTVAFLLGFVLDLI | SEQ ID NO: 120 |
| CA9 | | |
| Reverse | | |

TABLE 7
Non-limiting examples of pHLIP® sequences. A cysteine, a lysine,
an azido-modified amino acid, or an alkynyl modified amino
acid can be incorporated at the N-terminal (first 6 residues)
or C-terminal (last 6 residues) parts of the peptides
for conjugation with a cargo, and a linker. Italicized
residues are non-natural amino acids (see Table 4)
and are given in their 3 letter code.

| SEQ ID NO | Name | Sequence |
|---|---|---|
| SEQ ID NO: 121 | WT-2D | AEQNPIYWARYADWLFTTPLLLLDLALLVDADET |
| SEQ ID NO: 122 | WT-6E | AEQNPIYWARYAEWLFTTPLLLLELALLVEAEET |
| SEQ ID NO: 123 | WT-3D | ADDQNPWRAYLDLLFPDTTDLLLLDLLWDADET |
| SEQ ID NO: 124 | WT-9E | AEEQNPWRAYLELLFPETTELLLLELLWEAEET |
| SEQ ID NO: 125 | WT-GlaD | AEQNPIYWARYAGlaWLFTTPLLLLDLALLVDADET |
| SEQ ID NO: 126_ | WT-DGla | AEQNPIYWARYADWLFTTPLLLLGlaLALLVDADET |
| SEQ ID NO: 127 | WT-2Gla | AEQNPIYWARYAGlaWLFTTPLLLLGlaLALLVDADET |
| SEQ ID NO: 128 | WT-AadD | AEQNPIYWARYAAadWLFTTPLLLLDLALLVDADET |
| SEQ ID NO: 129 | WT-DAad | AEQNPIYWARYADWLFTTPLLLLAadLALLVDADET |
| SEQ ID NO: 130 | WT-2Aad | AEQNPIYWARYAAadWLFTTPLLLLAadLALLVDADET |
| SEQ ID NO: 131 | WT-GlaAad | AEQNPIYWARYAGlaWLFTTPLLLLAadLALLVDADET |
| SEQ ID NO: 132 | WT-AadGla | AEQNPIYWARYAAadWLFTTPLLLLGlaLALLVDADET |
| SEQ ID NO: 133 | WT-1 | GGEQNPIYWARYADWLFTTPLLLLDLALLVDADEGT |
| SEQ ID NO: 134 | WT-2 | GGEQNPIYWARYADWLFTTPLLLLDLALLVDADEGT |
| SEQ ID NO: 135 | WT-3 | AAEQNPIYWARYADWLFTTPLLLLDLALLVDADEGT |
| SEQ ID NO: 136 | WT-4 | AEQNPIYWARYADWLFTTPLLLLDLALLVDADEGT |
| SEQ ID NO: 137 | WT-2N | AEQNPIYWARYANVVLFTTPLLLLNLALLVDADEGT |
| SEQ ID NO: 138 | WT-2K | AEQNPIYWARYAKWLFTTPLLLLKLALLVDADEGT |
| SEQ ID NO: 139 | WT-2DNANQ | GGEQNPIYWARYADWLFTTPLLLLDLALLVNANQGT |
| SEQ ID NO: 140 | WT-D25A | AAEQNPIYWARYADWLFTTPLLLLALALLVDADEGT |
| SEQ ID NO: 141 | WT-D14A | AAEQNPIYWARYAAWLFTTPLLLLDLALLVDADEGT |
| SEQ ID NO: 142 | WT-P20A | AAEQNPIYWARYADWLFTTALLLLDLALLVDADEGT |
| SEQ ID NO: 143 | WT-D25E | AAEQNPIYWARYADWLFTTPLLLLELALLVDADEGT |
| SEQ ID NO: 144 | WT-D14E | AAEQNPIYWARYAEWLFTTPLLLLDLALLVDADEGT |
| SEQ ID NO: 145 | WT-3D-2 | AAEQNPIIYWARYADWLFTDLPLLLLDLLALLVDADEGT |
| SEQ ID NO: 146 | WT-R11Q | GEQNPIYWAQYADWLFTTPLLLLDLALLVDADEG |
| SEQ ID NO: 147 | WT-D25Up | GGEQNPIYWARYADWLFTTPLLLDLLALLVDADEG |
| SEQ ID NO: 148 | WT-D25Down | GGEQNPIYWARYADWLFTTPLLLLLDALLVDADEG |
| SEQ ID NO: 149 | WT-D14Up | GGEQNPIYWARYDAWLFTTPLLLLDLALLVDADEGT |
| SEQ ID NO: 150 | WT-D14Down | GGEQNPIYWARYAWDLFTTPLLLLDLALLVDADEG |
| SEQ ID NO: 151 | WT-P20G | AAEQNPIYWARYADWLFTTGLLLLDLALLVDADEGT |
| SEQ ID NO: 152 | WT-DH | DDDEDNPIYWARYADWLFTTPLLLLHGALLVDAD |
| SEQ ID NO: 153 | WT-2H | DDDEDNPIYWARYAHWLFTTPLLLLHGALLVDADE |
| SEQ ID NO: 154 | WT-L16H | CEQNPIYWARYADWHFTTPLLLLDLALLVDADE |
| SEQ ID NO: 155 | WT-1Wa | AEQNPIYWARYADFLFTTPLLLLDLALLVDADET |
| SEQ ID NO: 156 | WT-1Wb | AEQNPIYFARYADWLFTTPLLLLDLALLVDADE |
| SEQ ID NO: 157 | WT-1Wc | AEQNPIYFARYADFLFTTPLLLLDLALLWDADET |
| SEQ ID NO: 158 | WT-W6 | ADNNPWIYARYADLTTFPLLLLDLALLVDFDD |
| SEQ ID NO: 159 | WT-W17 | ADNNPFIYARYADLTTWPLLLLDLALLVDFDD |
| SEQ ID NO: 160 | WT-W30 | ADNNPFIYARYADLTTFPLLLLDLALLVDWDD |
| SEQ ID NO: 161 | WT-W17-P7 | ADNNPFPYARYADLTTWILLLLDLALLVDFDD |
| SEQ ID NO: 162 | WT-W39-R11 | ADNNPFIYAYRADLTTFPLLLLDLALLVDWDD |
| SEQ ID NO: 163 | WT-W30-R15 | ADNNPFIYATYADLRTFPLLLLDLALLVDWDD |
| SEQ ID NO: 164 | WT-Rev | Ac-TEDADVLLALDLLLLPTTFLWDAYRAWYPNQEA-Am |
| SEQ ID NO: 165 | Van-3D | AEDQNPYWARYADWLFTTPLLLLDLALLVD |
| SEQ ID NO: 166 | Var1-1D2E | AEDQNPYWARYADWLFTTPLLLLELALLVE |
| SEQ ID NO: 167 | Var2-3D | AEDQNPYWRAYADLFTPLTLLDLLALWD |
| SEQ ID NO: 168 | Var3-3D | ADDQNPWRAYLDLLFPTDTLLLDLLW |
| SEQ ID NO: 169 | Var3-WT | ADDQNPWRAYLDLLFPTDTLLLDLLWDADE |
| SEQ ID NO: 170 | Var3-Gla2D | ADDQNPWRAYLGlaLLFPTDTLLLDLLW |
| SEQ ID NO: 171 | Var3-DGlaD | ADDQNPWRAYLDLLFPTGlaTLLLDLLW |
| SEQ ID NO: 172 | Var3-2DGla | ADDQNPWRAYLDLLFPTDTLLLGlaLLW |
| SEQ ID NO: 173 | Var3-2GlaD | ADDQNPWRAYLGlaLLFPTGlaTLLLDLLW |
| SEQ ID NO: 174 | Var3-GlaDGla | ADDQNPWRAYLGlaLLFPTDTLLLGlaLLW |
| SEQ ID NO: 175 | Var3-D2Gla | ADDQNPWRAYLDLLFPTGlaTLLLGlaLLW |
| SEQ ID NO: 176 | Var3-3Gla | ADDQNPWRAYLGlaLLFPTGlaTLLLGlaLLW |
| SEQ ID NO: 177 | Var3-Aad2D | ADDQNPWRAYLAadLLFPTDTLLLDLLW |
| SEQ ID NO: 178 | Var3-DAadD | ADDQNPWRAYLDLLFPTAadTLLLDLLW |
| SEQ ID NO: 179 | Var3-2DAad | ADDQNPWRAYLDLLFPTDTLLLAadLLW |
| SEQ ID NO: 180 | Var3-2AadD | ADDQNPWRAYLAadLLFPTAadTLLLDLLW |
| SEQ ID NO: 181 | Var3-AadDAad | ADDQNPWRAYLAadLLFPTDTLLLAadLLW |
| SEQ ID NO: 182 | Var3-D2Aad | ADDQNPWRAYLDLLFPTAadTLLLAadLLW |
| SEQ ID NO: 183 | Var3-3Aad | ADDQNPWRAYLAadLLFPTAadTLLLAadLLW |
| SEQ ID NO: 184 | Var3-GlaAadD | ADDQNPWRAYLGlaLLFPTAadTLLLDLLW |
| SEQ ID NO: 185 | Var3-GlaDAad | ADDQNPWRAYLGlaLLFPTDTLLLAadLLW |
| SEQ ID NO: 186 | Var3-2GlaAad | ADDQNPWRAYLGlaLLFPTGlaTLLLAadLLW |
| SEQ ID NO: 187 | Var3-AadGlaD | ADDQNPWRAYLAadLLFPTGlaTLLLDLLW |
| SEQ ID NO: 188 | Var3-AadDGla | ADDQNPWRAYLAadLLFPTDTLLLGlaLLW |
| SEQ ID NO: 189 | Var3- | ADDQNPWRAYLGlaLLFPTAadTLLLGlaLLW |
| SEQ ID NO: 190 | GlaAadGla | GEEQNPWLGAYLDLLFPLELLGLLELGLW |
| SEQ ID NO: 191 | Var3-GLL | ADDDDDDPWQAYLDLLFPTDTLLLDLLW |
| SEQ ID NO: 192 | Var3-M | AEEQNPWRAYLELLFPTETLLLELLW |
| SEQ ID NO: 193 | Var4-3E | ADDQNPWARYLDWLFPTDTLLLDL |
| SEQ ID NO: 194 | Var5-3Da | DNNNPWRAYLDLLFPTDTLLLDW |
| SEQ ID NO: 195 | Var6-3Db | AEEQNPWARYLEWLFPFETLLLEL |
| SEQ ID NO: 196 | Var7-3E | DDDDDDPWQAYLDLFPTDTLALDLW |
| SEQ ID NO: 197 | Var7-M | EEQQPWAQYLELLFPFETLLLEW |
| SEQ ID NO: 198 | Var8-3E | EEQQPWRAYLELLFPTETLLLEW |
| SEQ ID NO: 199 | Vat9-3E | AEDQNPWARYADWLFPTTLLLLD |
| SEQ ID NO: 200 | Var10-2D | AEEQNPWARYAEWLFPTTLLLLE |
| SEQ ID NO: 201 | Var11-2E | AEDQNPWARYADLLFPTTLAW |
| SEQ ID NO: 202 | Var12-1D | AEEQNPWARYAELLFPTTLAW |
| SEQ ID NO: 203 | Var13-1E | DDDDDNPNYWARYANVVLETTPLLLLNGALLVEAEET |
| SEQ ID NO: 204 | Var15-2N | DDDDDNPNYWARYAPWLFTTPLLLLPGALLVEAEET |
| SEQ ID NO: 205 | Var16-2P | AEQNPIYFARYADFLFTTPLLLLDLALLWDADET |
| SEQ ID NO: 206 | Var17 | AEQNPIYWARYADFLFTTPLLLLDLALLVDADET |
| SEQ ID NO: 207 | Var18 | AEQNPIYWARYADWLFTTPL |
| SEQ ID NO: 208 | Var19a | AEQNPIYFARYADLLEPTTLAW |
| SEQ ID NO: 209 | Var20 | AEQNPIYWARYADLLFPTTLAF |
| SEQ ID NO: 210 | Var21 | AEQNPIYWARYADLLFPTTLAW |
| SEQ ID NO: 211 | Var22 | AEQNPIYFARYADWLETTPL |
| SEQ ID NO: 212 | Var23 | EDQNPWARYADLLFPTTLAW |
| SEQ ID NO: 213 | Var24 | GLAGLAGLLGLEGLLGLPLGLLEGLWLGLELEGN |
| SEQ ID NO: 214 | ATRAM | EQNPIYILDLVEGLLFAVTSVDELVQWDDAGD |
| SEQ ID NO: 215 | pHLIP-CA9 | NLEGFFATLGGEIALWSLVVLAIE |
| SEQ ID NO: 216 | pHLIP-Rho | NNEGFFATLGGEIALWSDVVLAIE |
| SEQ ID NO: 217 | pHLIP-RhoM1 | DNNEGFFATLGGEIPLWSDVVLAIE |
| | pHLIP-RhoM2 | |

Corticosteroid—pHLIP® Constructs

An exemplary composition is characterized by the following formula:

A-M-Linker-CS (1),

wherein:

“A” is a pHLIP®. pHLIP® peptides are described here and in U.S. Pat. Nos. 9,814,781 and 9,289,508 (hereby incorporated by reference) as well as U.S. Patent Publication 20180117183, 20180064648, 20180221500, 20180117183, 20180064648, 20160256560, 20150191508, 20150051153, and 20120142042, 20120039990, and 20080233107, each of which is hereby incorporated by reference.

In other examples, the composition is characterized by the following structure: A-L-Cs, wherein “A” is a pHLIP® peptide; “L” is a polyethylene glycol linker; “Cs” is a corticosteroid, and wherein each “-” is a covalent bond.

“M” is a polar modulator, and it is optional. It comprises a chemical entity to modulate the overall polarity of the Linker-CS moiety and solubility of the entire construct for optimized targeting by pHLIP®. To achieve optimized targeting, the overall polarity of M-Linker-CS is measured by Log P, where P is the measured octanol-water partition coefficient. For delivery, Log P is preferably in the range −1<Log P<1. Polar means: Log P<−0.4; Moderately hydrophobic: 2.5<Log P<−0.4; and Hydrophobic: Log P>2.5. The polarity and/or hydrophobicity of a drug or compound to be delivered is measured using methods known in the art, e.g., by determining Log P, in which P is octanol-water partition coefficient. A substance is dissolved into octanol-water mixture, mixed and allowed to come to equilibration. The amount of substance in each (or one) phases is then measured. The measurements can be done in a number of ways known in the art, e.g., by measuring absorbance, or determining the compound amounts using NMR, HPLC, or other known methods.

“L” is a linker, which can range from relatively small, e.g., only a few atoms, to a rather large polymer of 4-5 kDa. An exemplary heterobifunctional linker that reacts on one end with a free thiol to spontaneously form a disulfide bond, with thiopyridine as a leaving group, and on the other end reacts with activated amine or hydroxyl groups in the presence of DIPEA, and in some cases DMAP or other activator base, to form a carbamate or carbonate, respectively. This material can be used if pHLIP® (A) is protected at its amino terminus, such as with N-acetylation. This material can also be reacted with pHLIP® bearing a cysteine residue or with a thiol-bearing linker for subsequent conjugation to pHLIP®, and forms a conjugate by disulfide exchange with thiopyridine as a leaving group. This material can be used to form a conjugate with pHLIP® bearing a lysine residue, if pHLIP® is protected at its amino terminus, such as with N-acetylation.

An exemplary heterobifunctional linker:

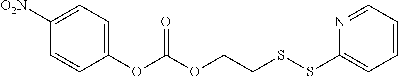 [SMILES: O=C(OCCSSc1ccccn1)Oc1ccc([N+](=O)[O-])cc1]

A diagram of a pHLIP construct with a linker:

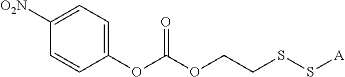

A diagram of a linker and a CS for S—S exchange:

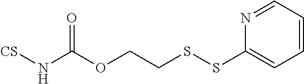

A diagram of a linker and a CS for conjugation with primary amines:

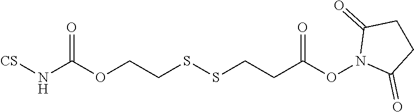

In some examples, the following cross-linkers can be used: SPDP (succinimidyl 3-(2-pyridyldithio)propionate); LC-SPDP (succinimidyl 6-(3(2-pyridyldithio)propionamido)hexanoate); sulfo-LC-SPDP (sulfosuccinimidyl 6-(3′-(2-pyridyldithio)propionamido)hexanoate); PEG4-SPDP (PEGylated, long-chain SPDP crosslinker); PEG12-SPDP (PEGylated, long-chain SPDP crosslinker); SMCC (succinimidyl 4-(N-maleimidomethyl)cyclohexane-1-carboxylate); sulfo-SMCC (sulfosuccinimidyl 4-(N-maleimidomethyl)cyclohexane-1-carboxylate); SMPT (4-succinimidyloxycarbonyl-alpha-methyl-α(2-pyridyldithio)toluene); DTME (dithiobismaleimidoethane). The invention may encompass the following embodiments.

A Linker comprises a covalent bond or a chemical connection such that (1) is selected from the following, where Drug is a corticosteroid:

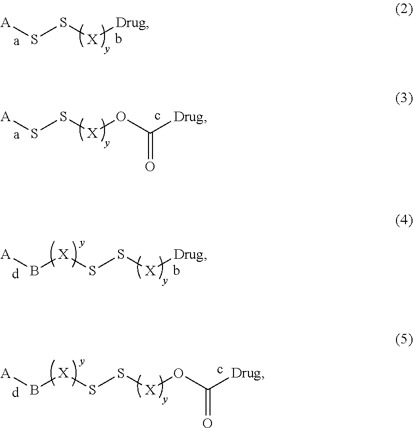

each occurrence of y may be present or absent and is independently an integer ranging from 1 to 4;

each occurrence of X is independently selected from the group consisting of CH₂, CH(alkyl), and C(alkyl)₂;

each occurrence of B may be present or absent and is independently selected from the group consisting of alkyl, aryl, and PEG;

bond a is formed between the sulfur and the thiol substituent of a cysteine residue in A;

bond b is formed between the carbon and a substituent on the Drug (corticosteroid), wherein the substituent is selected from the group consisting of hydroxyl, carbonyl, amine, amide, sulfate, sulfonamide, phosphate, and phosphoramide;

bond c is formed between the carbonyl and a substituent on Drug (corticosteroid), wherein the substituent is selected from the group consisting of primary amine, secondary amine, and hydroxyl;

bond d is formed between B and an amino acid residue in A, wherein the amino acid is selected from the group consisting of serine, threonine, tyrosine, tryptophan, histidine, lysine, and cysteine and comprises an amide, ester, carbamate, carbonate, or maleimide bond.

Non-limiting examples of corticosteroids include Flugestone (flurogestone), Fluorometholone, Medrysone (hydroxymethylprogesterone), Prebediolone acetate (21-acetoxypregnenolone), chlormadinone acetate, cyproterone acetate, Medrogestone, Medroxyprogesterone acetate, Megestrol acetate, Segesterone acetate, Chloroprednisone, Cloprednol, Difluprednate, Fludrocortisone, Fluocinolone, Fluperolone, Fluprednisolone, Loteprednol, Methylprednisolone, Prednicarbate, Prednisolone, Prednisone, Tixocortol, Triamcinolone, Alclometasone, Beclometasone, Betamethasone, Clobetasol, Clobetasone, Clocortolone, Desoximetasone, Dexamethasone, Dexamethasone phosphate, Diflorasone, Difluocortolone, Fluclorolone, Flumetasone, Fluocortin, Fluocortolone, Fluprednidene, Fluticasone, Fluticasone furoate, Halometasone, Meprednisone, Mometasone, Mometasone furoate, Paramethasone, Prednylidene, Rimexolone, Ulobetasol (halobetasol), Amcinonide, Budesonide, Ciclesonide, Deflazacort, Desonide, Formocortal (fluoroformylone), Fluclorolone acetonide (flucloronide), Fludroxycortide (flurandrenolone, flurandrenolide), Flunisolide, Fluocinolone acetonide, Fluocinonide, Halcinonide, Triamcinolone acetonide, Cortivazol, and RU-28362, as well as derivatives and analogs thereof.

A compound of formula (1), wherein CS is selected from a group of potent corticosteroids.

A compound of formula (1), e.g., wherein CS is dexamethasone or betamethasone and their analogs and derivatives.

### Dexamethasone:

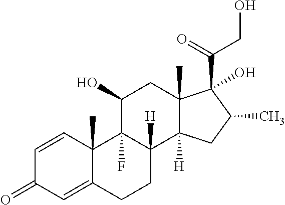

### Betamethasone:

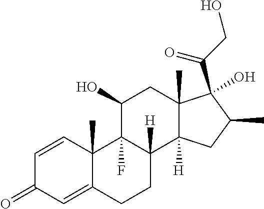

Non-limiting examples of dexamethasone analogs and derivatives include dexamethasone phosphate:

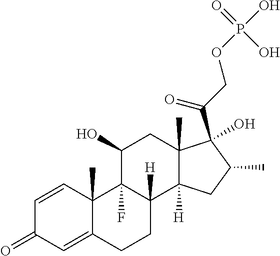

dexamethasone phosphate ester:

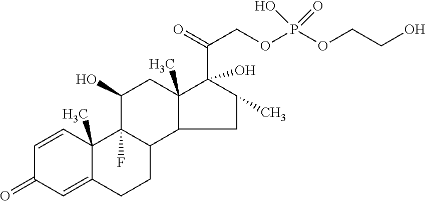

dexamethasone hemisuccinate:

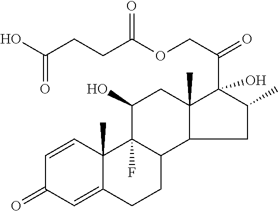

dexamethasone glucuronide:

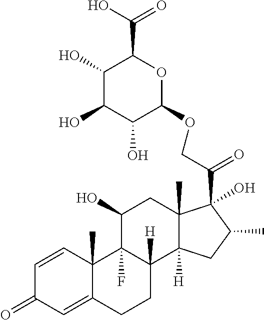

Monocarboxyl and monoamine analogs of corticosteroids can be synthesized. Carboxylation of corticosteroids occurs through the introduction of glutarate, hemisuccinate, or —(O-carboxymethyl)oxime and can potentially occur at hydroxyl groups located at the C3, C6, C11, C17, or C21 positions (C21 is preferable).

Monoamine derivatives of corticosteroids occur by utilizing N-trityl-glycine and a carbodiimide resulting in the production of a trityl-glycine-steroid intermediate that is then converted by AcOH to a glycyl corticosteroid. The monoamine or monocarboxyl group of the corticosteroid is transformed into a covalent amide bond. Given this general synthesis strategy, corticosteroids initially can be converted to either a monoamine or a monocarboxyl analog that is then covalently bound to a primary carboxyl group or primary amine group, respectively, on a pHLIP® peptide via cleavable or non-cleavable links.

pHLIP® peptides improve and expand the clinical use of corticosteroids by providing an efficient and reliable method to treat cells/tissues in need of treatment while leaving cells not in need of treatment substantially unaffected by the drug. pHLIP® peptides target corticosteroids to cell surface acidity in inflamed tissues, where they translocate corticosteroids across plasma membranes into the cytoplasms of cells, thereby suppressing inflammation in the targeted tissues while avoiding the side effects resulting from non-targeted administration.

### Therapeutic Methods—Corticosteroids

The amount of corticosteroid (e.g., dexamethasone or betamethasone) to be administered to a subject depends upon the particular corticosteroid used.

The dose typically used in mice in the studies described herein was 4.6 mg/kg of pHLIP-Dexa. In this dose of the pHLIP®-corticosteroid construct, it was 0.46 mg/kg Dexa (the rest is pHLIP®). To calculate/translate the dose from mice to humans, 0.46 mg/kg Dexa (within pHLIP®-Dexa) in mice corresponds to 0.0368 mg/kg in humans. As is well known in the art, the human dose is typically calculated for a 70 kg individual, i.e., 2.6 mg of Dexa (active ingredient) within 26 mg of pHLIP®-Dexa. This dose has been demonstrated to work. For example, an exemplary human dose of pHLIP-Dexa is 2.6 mg per injection of active ingredient (corticosteroid), e.g., Dexa. As is known in the art, the dose can be adjusted by a physician in view of weight and/or medical condition of the individual to be treated.

In general, the amount is selected so as to maximize the therapeutic benefit while minimizing systemic absorption and adverse side effects, e.g., systemic (or specific) side effects. A daily dosage amount of corticosteroid can be less than or equal to 50 mg. In one embodiment the amount of corticosteroid is between about 0.01 mg and about 20 mg. I. In still another embodiment the amount of corticosteroid is between about 2 mg and about 10 mg. In yet another example the amount of corticosteroid is between about 2 mg and about 5 mg. In still other embodiments, the amount of corticosteroid is about 0.01 mg, about 0.02 mg, about 0.03 mg, about 0.04 mg, about 0.05 mg, about 0.06 mg, about 0.07 mg, about 0.08 mg, about 0.09 mg, about 0.1 mg, about 0.2 mg, about 0.3 mg, about 0.4 mg, about 0.5 mg, about 1 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 90 mg, or about 20 mg, inclusive of all ranges and subranges there between.

For example, adults may be orally administered dexamethasone in a range from 0.75 to 9 milligrams (ma) per day, and children (e.g., <12 years of age) may be administered 0.02 to 0.3 mg per kilogram (kg) of body weight per day, divided and taken 3 or 4 times a day. Furthermore, the intravenous (IV) or intramuscular (IM) dosage for dexamethasone may be 0.5 to 9 mg/day IV or IM, divided every 6 to 12 hours for adults and 0.02 to 0.3 mg/kg/day IV or IM given in 3 to 4 divided doses for infants, children and adolescents.

For example, dexamethasone is used for treatment of multiple myelomas in combination with chemotherapeutic agents. Presently (prior to the invention), an exemplary dose of dexamethasone for treatment of myeloma is 20 mg taken orally each second day 8 times or 40 mg taken orally each 8^{th} days for 4 times in the course of one treatment. However as described herein, pHLIP® conjugated corticosteroid compositions can reduce the dose with the advantage of reducing adverse side effects. Moreover, combination therapy in which both a corticosteroid is conjugated to pHLIP® and a chemotherapeutic agent (e.g., calicheamicin) is conjugated to pHLIP® (pHLIP®-calicheamicin) further reduces dosages required for clinical benefit.

Calicheamicins belong to a class of enediyne antitumor antibiotics derived from the bacterium Micromonospora echinospora. In addition to calicheamicin, various chemotherapeutic agents are useful for combination therapy. Non-limiting examples include alkylating agents (such as nitrogen mustards, notrisoureas, alkyl sulfonates, triazines, ethylenimines, and platinum-based compounds); antimetabolites (such as 5-fluorouracil (5-FU), 6-mercaptopurine (6-MP), capecitabine (Xeloda®), cytarabine (Ara-C®), floxuridine, fludarabine, gemcitabine (Gemzar®), hydroxyurea, methotrexate, and pemetrexed (Alimta®)); topoisomerase inhibitors (e.g., topotecan, irinotecan, etoposide, and teniposide); taxanes (such as paclitaxel and docetaxel); platinum-based chemotherapeutics (such as cisplatin and carboplatin); anthracyclines (such as daunorubicin, doxorubicin (Adriamycin®), epirubicin, and idarubicin); epothilones (e.g., ixabepilone); vinca alkaloids (e.g., vinblastine (Velban®), vincristine (Oncovin®), and vinorelbine (Navelbine®)); estramustine; actinomycin-D; mitomycin-C; mitoxantrone; imatinib; lenalidomide; pemetrexed; bortezomib; leuprorelin; and abiraterone. Other chemotherapeutic acids for treatment are well know in the art. Use of a pHLIP®-corticosteroid in a combination therapy regimen with a pHLIP®-chemotherapeutic agent leads to a synergistic effect in treatment of cancer.

In adults, betamethasone is typically administered orally in a range from 0.6-7.2 mg orally divided twice daily/four times daily or 0.6-9 mg/day intramuscularly each day divided twice daily. A pediatric dose for children under 12 years old ranges from 0.0175-0.25 mg/kg/day intramuscular or orally divided every 6-12 hours. Local injections of betamethasone in adults may be from 4 to 8 mg and children may require smaller doses. As is described above, use of pHLIP-corticosteroid construct/conjugate permits a reduction of the dose of corticosteroid and resultant reduction in adverse side effects.

Thus, the amount of the corticosteroid (e.g., dexamethasone or betamethasone) for therapeutic benefit can be decreased when administered in combination with a pHLIP® peptide, e.g., pHLIP-Dexa. For example, the amount may decrease by about 5%, 10%, 20% 30%, 40% 50% 75%, 2-fold, 3-fold, 5-fold, 7-fold, 10-fold or more compared to the level of the corticosteroid alone.

Corticosteroids are typically given to a subject by parenteral administration [intravenous (IV) or intramuscular (IM)], e.g., Dexa, one of the most potent steroids, is often administered IV. However, corticosteroids are also administered intramuscularly, topically, locally (e.g., direct inject to an affected anatomical location such as lung or joint, e.g., articulating joint such as knee, hip, shoulder, elbow, spine/vertebra) and other clinically acceptable routes of administration. In preferred embodiments, subjects are treated with pHLIP-Dexa, using IV or local administration.

A significant advantage of the invention is that the corticosteroid-pHLIP constructs, e.g., pHLIP-Dexa are associated with few adverse or deleterious side effects of conventional corticosteroid therapies. For example, without pHLIP®, a corticosteroid, e.g, dexamethasone can cross the blood-brain barrier and lead to adverse side effects such as aberrant cortisol levels, mood changes, vision changes, and/or insomnia. Other side effects include swelling, rapid weight gain, acne, dry skin, thinning skin, stomach upset, nausea, vomiting, increase hair growth, and/or skin rash.

pHLIP® does not deliver corticosteroids to the brain, thereby avoiding aberrant cortisol levels, mood changes, vision changes, insomnia, depression, headache, dizziness, anxiety, and/or agitation. Moreover, convention corticosteroid therapy can often lead to systemic dissemination of the drug which can lead to other adverse side effects described. The pHLIP-corticosteroid constructs described herein preferentially and specifically deliver the corticosteroid locally, e.g., and inflamed joint or lung or kidney or liver or other organs, while avoiding systemic dissemination and systemic metabolic effect

The efficiency of the pHLIP-corticosteriod is also more efficient and effective than conventional delivery approaches as demonstrated herein. The mouse model of lung inflammation induced by bleomycin is one of the harshest and extreme models of inflammation known. For example, pHLIP-Dexamethasone effectively reduced histological inflammation in a very extreme model of inflammation, whereas dexamethasone along had little or no effect on inflammation in this severe model.

### Evaluation of Inflammation

Evaluation of inflammation in mammalian subject, e.g., human patients, is well known in the art. Five classical signs of inflammation are heat, pain, redness, swelling, and loss of function. Such signs are useful in evaluating local inflammation of tissues, e.g., inflammation of articulating joints. Blood tests for inflammation include Erythrocyte sedimentation rate (ESR), C-reactive protein (CRP) and plasma viscosity (PV) blood tests. In addition to CRP measurements, there are a number of available parameters for inflammation diagnostics, including procalcitonin, leukocyte, or a change in thrombocyte counts. See, e.g., Hoffmann, et al. “Diagnostic testing for a high-grade inflammation: parameter dynamics and novel markers,” CLin Chem Lab Med 2015; 53(4): p. 541-547, incorporated by reference in its entirety (“Hoffmann”). Moreover, the granularity index has been shown to be a marker of leukocyte activation, quantifies toxic granulation in neutrophils. The granularity of the neutrophils can be determined by sideward-scattered light measurements with Sysmex hematology analyzers. See Hoffmann at p. 541, col. 2. Another diagnostic measure also includes cytokine-induced activation of hepcidin expression, which measures iron redistribution during inflammation. Id. at p. 542, col. 1.

### Examples

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: Evaluation of ICG-pHLIP Targeting of Inflamed Lungs in a Mouse LPS-Induced Model of Pulmonary Inflammation

The main goal of this study demonstrated the ability of pHLIP® to target inflamed tissue. Inflammation in lungs is induced by LPS (Lipopolysaccharides from E. coli 0111: B4) challenge, which is well-established model of inflammation. CD-1 male mouse, 6 to 7 weeks and 32-38 grams from Charles River Labs are used in the study. Mice were anesthetized by ketamine hydrochloride/xylazine hydrochloride by intraperitoneal injection and allowed 5 minutes to reach maximal anesthesia. For intranasal administration of LPS mice were suspended by the upper jaw, such that the nose and trachea were lined up vertically directly above the bronchi, to allow optimal inhalation and passive gravitational flow of LPS directly to the lungs. 50 μg (in 50 μl) LPS were instilled into the nares in five boluses of 10 μl each. Mice were maintained in their vertical orientation for 5 minutes following the last bolus, to allow fullest penetration of LPS to the lungs before signs of awakening. After 3 hours of inoculation with the LPS vehicle (PBS) or fluorescent ICG-pHLIP (0.5 mg/kg) were administrated as a single intraperitoneal injection. Animals were sacrificed after 2 and 21 hours. Liver, both kidneys and gastrocnemius muscles were dissected and snap frozen for imaging. Imaging was performed using near infrared imager.

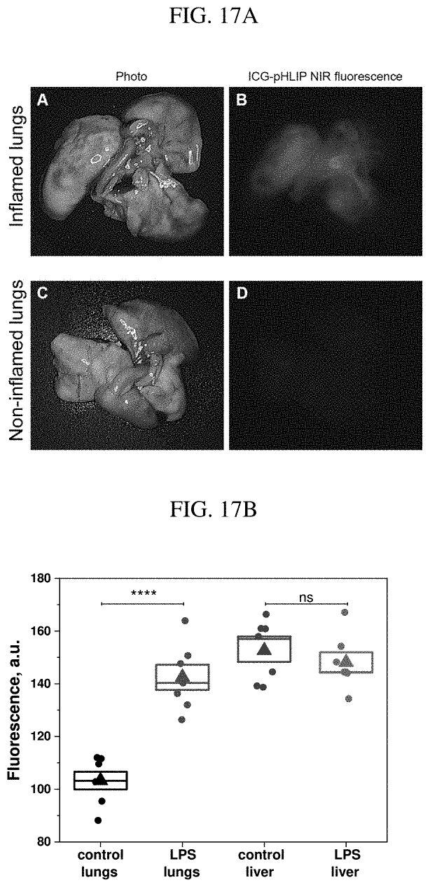

The obtained data indicated that ICG-pHLIP targeted acidic inflamed lungs, while a very low signal was observed in animals with non-inflamed lungs 21 hrs after construct administration (FIG. 17A). At the same time, the fluorescent signal in the liver from animals challenged with LPS and control animals was similar (FIG. 17B), thereby confirming specific accumulation of ICG-pHLIP® in inflamed lungs of animals challenged with LPS.

### Example 2: Evaluation of pHLIP-Dexa Treatment of Inflamed Lungs in a Mouse LPS-Induced Model of Pulmonary Inflammation

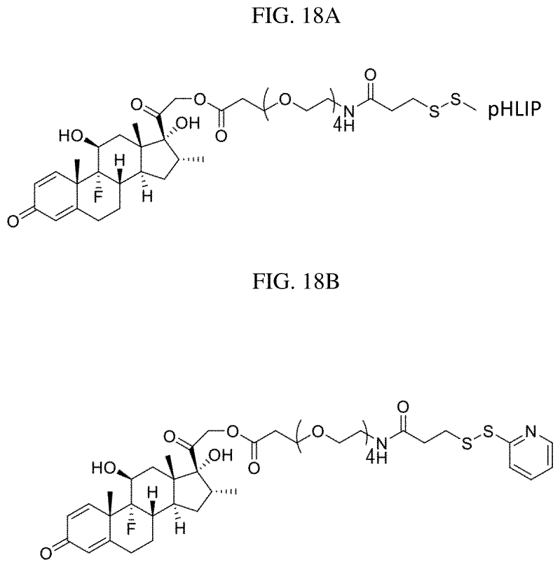

Since inflamed lungs were targeted by pHLIP®, pHLIP-Dexa was evaluated, where Dexa is a dexamethasone, to suppress inflammation in lungs. Dexamethasone-propionyl-PEG(4)-SPDP (Dexa-PEG4-SPDP) (FIG. 18A) was synthesized and purified by Iris Biotech. pHLIP-Dexa (FIG. 18B) was prepared by conjugation of pHLIP® (Var3) with single Cys residue at the C-terminal inserting end with Dexa-PEG4-SPDP. The Var3 pHLIP® peptide used in the study: ADDQNPWRAYLDLLFPTDTLLLDLLWCA (SEQ ID NO: 3) was prepared by solid-phase synthesis. The peptide and Dexa-PEG4-SPDP were dissolved in DMSO and mixed to have molar ratio 1:1. 100 mM sodium phosphate, 150 mM NaCl buffer, pH 7.2 (saturated with argon is added to reaction mix ( 1/10 of total volume). Reaction mixture was incubated at room temperature for 2 hours and the reaction progress was monitored by the analytical reverse phase HPLC (Zorbax SB-C18 column (4.6×250 mm, 5 μm; Agilent Technologies; the gradient of binary solvent system using water and acetonitrile with 0.05% TFA for 20-70% over 30 min). pHLIP—was purified by the reverse phase HPLC (Zorbax SB-C18 columns (9.4×250 mm, 5 μm; Agilent Technologies, the same gradient, over 30 min), lyophilized and characterized by SELDI-TOF mass spectrometry.

CD-1 male mouse, 6 to 7 weeks and 32-38 grams from Charles River Labs were used in the study. LPS challenge procedure was carried out on mice, as described in the Example 1, above. Briefly, mice were allowed optimal inhalation and passive gravitational flow of LPS directly to the lungs. 50 μg (in 50 μl) LPS was instilled into the nares in five boluses of 10 μl each. 30 minutes after inoculation with the LPS, mice were injected with vehicle (PBS/5% DMSO) and pHLIP-Dexa in PBS/5% DMSO at doses of 1.4, 4.6 or 11.4 mg/kg. Animals were sacrificed after two hours and 3.5 hours after administration with pHLIP-Dexa, serum and lung tissue are processed for determination of MCP-1 (monocyte chemoattractant protein) level, which is a known marker for inflammation. For processing, a mammalian protease inhibitor cocktail (diluted 1:100 in PBS) was added to each lung sample. Lungs were homogenized by Beadbeater with 2 mm zirconia beads for 1.5 minutes. Homogenates were spun at 10K for 10 minutes at 4° C., and supernatants collected for analysis. Quantikine kit (R&D Systems) assays for JE/MCP-1 were performed as per kit protocols. For the lungs samples, MCP-1 content was expressed as pg/mg of protein. Protein measurements were performed by Pierce™ BCA Protein Assay Kit according to the protocol provided by a manufacturer.

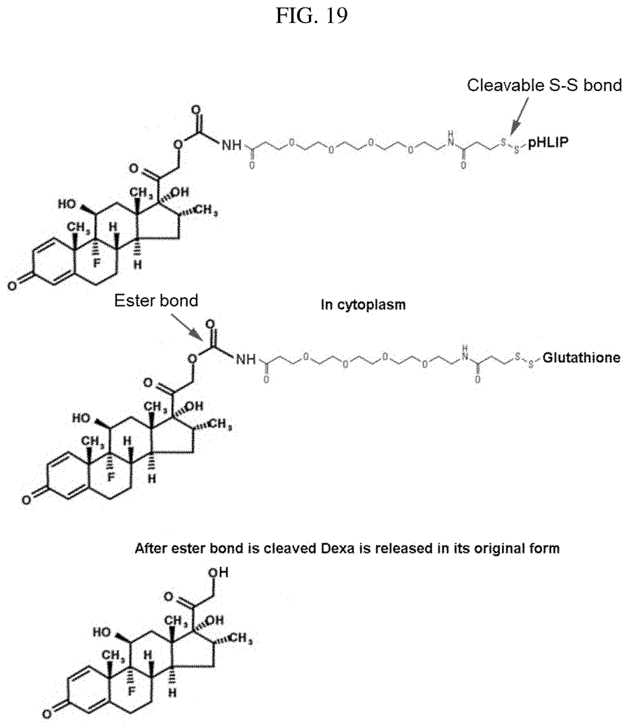

FIG. 19 demonstrates steps in cleavage of S—S and ester bonds to release Dexa in its original, non-modified form in cytoplasm of cells. According to the obtained data (FIG. 20), all cleavage steps occurred and Dexa was released in cells of inflamed lungs. The dose of 4.6 mg/kg of pHLIP-Dexa was very effective in reducing level of MCP-1 protein. The high concentration of pHLIP-Dexa was less effective due to aggregation issues (the solution of 11.4 mg/kg pHLIP-Dexa in PBS/5% DMSO is cloudy).

### Example 3: Evaluation of pHLIP-Dexa Treatment of Inflamed and Injured Lungs in a Mouse Bleomycin-Induced Lung Injury Model

Intranasal administration of bleomycin induces a significant pulmonary inflammation in mice, which typically progresses to fibrosis and eventually leads to mice death. pHLIP-Dexa was evaluated for the ability to reduce bleomycin-induced inflammation in lungs.

CD-1 male mouse, 6 to 7 weeks and 32-38 grams from Charles River Labs were used in the study. Bleomycin sulfate (4 Units/kg) in 50 μL (a solution of 2 Units/ml for 25 g mouse) was prepared in 0.9% NaCl. Bleomycin or saline was intranasally dosed on Day 0. pHLIP-Dexa in PBS/5% DMSO (4.6 mg/kg), Dexa (dexamethasone alone at dose of 0.46 mg/kg, which corresponds to the same number of molecules of Dexa in pHLIP-Dexa) or vehicle (PBS/5% DMSO) were dosed daily from day 0 to day 7. On Day 0, 3, 5 and 8 blood glucose was measured and body weights are recorded. On Day 8, all mice were euthanized. Lung tissues were collected and processed for MCP-1 analysis and part of the tissues was fixed in 10% formalin for histopathology. For processing of lungs for MCP-1 measurements the mammalian protease inhibitor cocktail (diluted 1:100 in PBS) was added to each lung sample. Lungs were homogenized by Beadbeater with 2 mm zirconia beads for 1.5 minutes. Homogenates were spun at 10K for 10 minutes at 4° C., and supernatants collected for analysis and frozen. Quantikine kits (R&D Systems) for JE/MCP-1 were run as per kit protocols. Formalin-fixed lung samples are paraffin-embedded, sectioned, processed for hemolysin and eosin (HE) staining and imaged. Ten 20× fields from each mouse were scored individually by a pathologist blinded to experimental treatments. Inflammation and hemorrhage were scored from 0-5 where 0 is normal, 1 is minimal change, 2 is mild change, 3 is moderate change, 4 is marked change, and 5 is severe change.

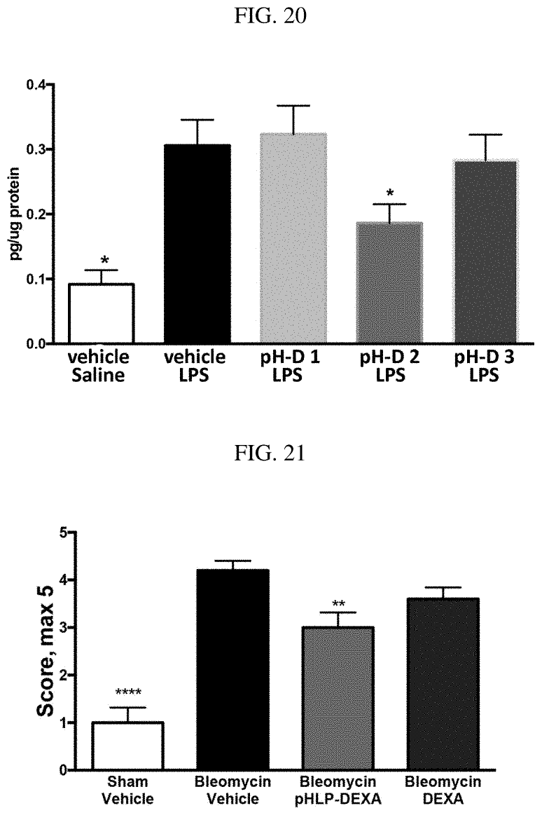

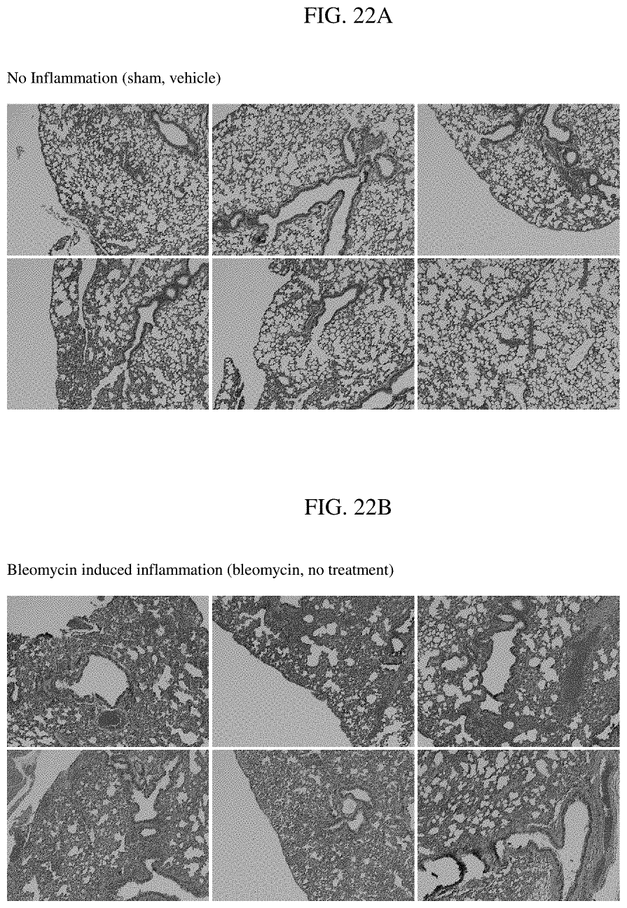

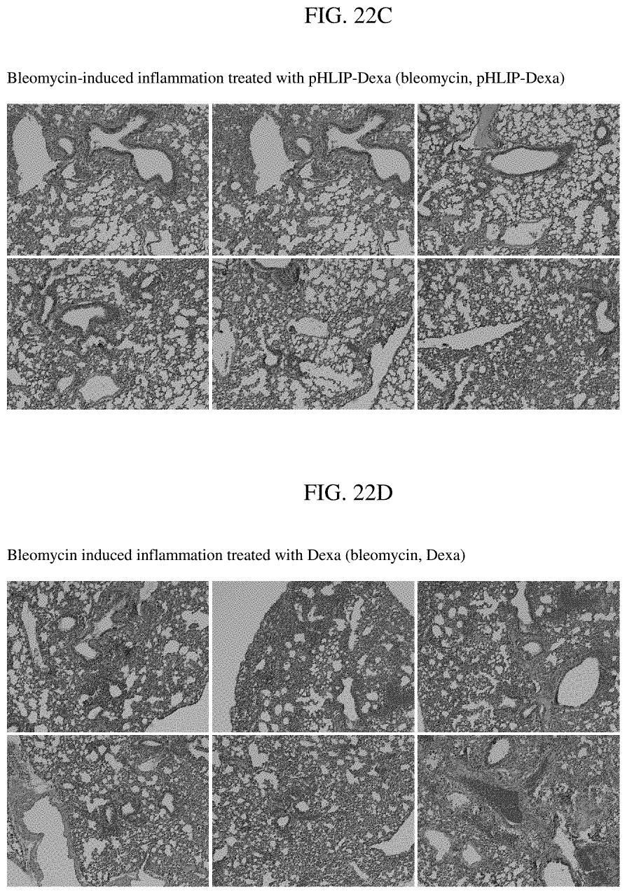

Bleomycin-induced lung injury is associated with irreversible changes in lungs, which eventually leads to animal death. Dexa alone administrated at concentration equivalent to Dexa in pHLIP-Dexa construct failed to exhibit statistically significant reduction of inflammation score assessed by HE analysis. The lungs remained significantly inflamed as is clearly seen from FIG. 22D compared to FIG. 22B and FIG. 22A. At the same time pHLIP-Dexa exhibited statistically significant reduction of inflammation (FIG. 21 and FIG. 22C vs FIG. 22B).

### Example 4: Evaluation of pHLIP-Dexa in Treatment of Arthritis in in a Mouse Model of Collagen-Induced Arthritis

pHLIP-Dexa was evaluated on collagen-induced model of arthritis. In this model of arthritis, inoculation of mice with collagen and adjuvant produces significant arthritic signs after 4 weeks and peaked between 33 and 42 days post-inoculation.

DBA male mouse, 9-10 weeks from Envigo were used in the study. Bovine collagen II (CII, 2 mg/ml) was mixed with an equal volume of complete Freund's adjuvant (CFA, 5 mg/mL M. tuberculosis) with a hand-held homogenizer on ice for several minutes until a stiff emulsion was achieved. Mice were injected with the CII/CFA emulsion according to the scheme:

Day 0—Immediately following the preparation of the CII/CFA emulsion the mice were dosed with 100 μl intradermally into the base of the tail with a 25 g needle.
Day 21 (Booster)—Immediately following the preparation of the CII/CFA emulsion the mice are dosed with 100 μl intradermally at the base of the tail with a 25 g needle. Between the initial and booster injections, mice are exposed to minimum handling.
Day 27—The animals were randomized by disease score and body weight into the dosing groups.
Day 28-42—Animals were dosed daily with vehicle (PBS/5% DMSO), dexamethasone (20 mg/kg and 0.46 mg/kg) and pHLIP-Dexa (4.6 mg/kg). pHLIP-DEXA (4.6 mg/kg) and low dose Dexa (0.46 mg/kg) were administered intraperitonially every other day for 14 days (7 injections). Vehicle and high dose of Dexa (20 mg/kg) were given orally daily for 14 days. Clinical scoring was performed for each of the 4 paws for a total possible score of 20 per mouse.

Clinical scores were evaluated using the following scale: 0—no clinical signs, normal; 1—hind or forepaw joint affected or minimal diffuse erythemia/swelling; 2—hind or forepaw joints affected or mild diffuse erythemia/swelling; 3—hind or forepaw joints affected or moderate diffuse erythemia/swelling; 4—marked diffuse erythemia/swelling or digit joints affected, severe diffuse erythemia/swelling of the entire paw, unable to flex digits.

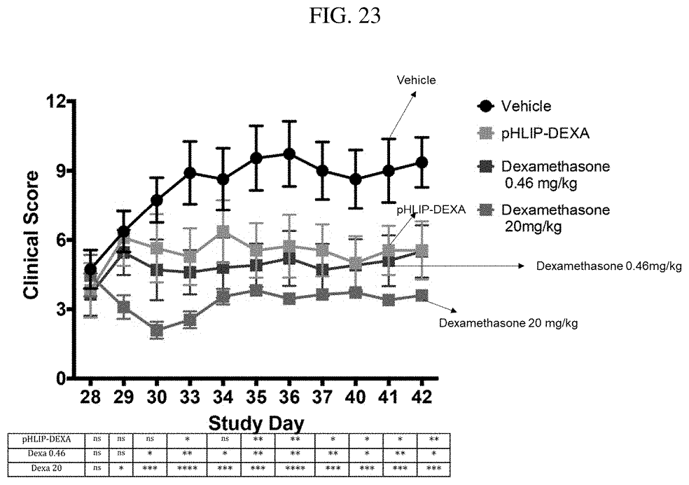

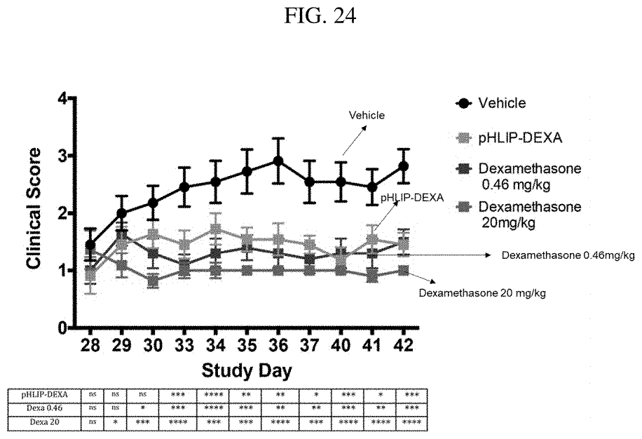

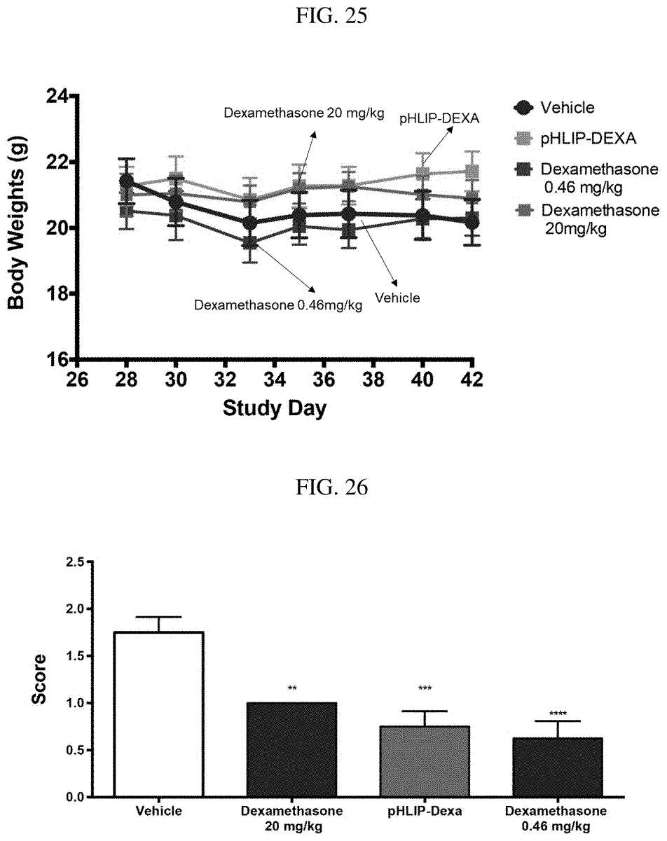

The total and average hid paw clinical scores for arthritis are statistically significantly reduced by treatment with pHLIP-Dexa and Dexa (FIG. 23 and FIG. 24) indicating effectiveness of pHLIP-Dexa treatment of arthritis. The least changes of body weight were observed for the group treated with pHLIP-Dexa.

### Example 5: Evaluation of pHLIP-Dexa in Treatment of Dermatitis in a Mouse Model of Contact Hypersensitivity (Dermatitis)

Oxazolone produces an increase in contact hypersensitivity as measured by ear thickness and visual score (erythema). Contact hypersensitivity model resembles progression of dermatitis in humans. pHLIP-Dexa was evaluated for the reduction of inflammation induced by oxazolone.

CD-1 male mouse, 8 to 10 weeks from Charles River Labs were used in the study. Contact hypersensitivity was induced by sensitization and challenge with oxazalone as follows: on day 1, mice are sensitized to oxazalone by application (50 μL; 3% in 100% ethanol) to the abdominal area (previously shaved) and to each footpad (5 μL). On day 6, mice were exposed to oxazalone (20 μL; 1% in 100% ethanol) by application to each side of right ear. Twenty-four hrs after challenge (day 7), dermatitis severity was measured using a visual scale: 0—no evidence of erythema or swelling; 1—mild swelling; 2—mild erythema and moderate selling; 3—erythema and severe swelling. Ear thickness was measured using a micrometer once, prior to sensitization on day 1 (baseline) and once, after dosing on day 7 of the study. Vehicle (PBS/5% DMSO), pHLIP-Dexa (4.6 mg/kg) and low dose of Dexa (0.46 mg/kg) are administrated intraperitonially, and high dose of Dexa (20 mg/kg) were administered orally daily 30 minutes prior to visual scoring and micrometer measurements.

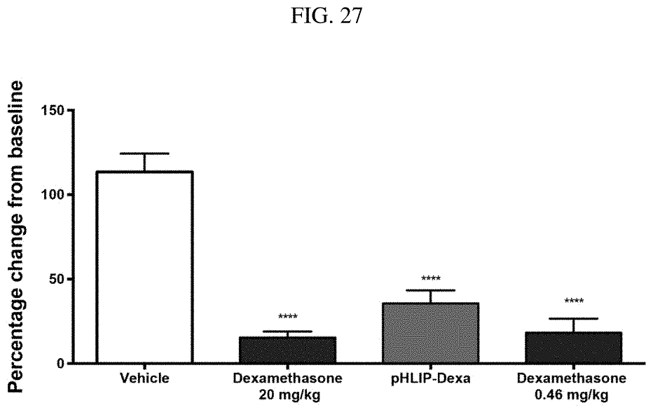

pHLIP-Dexa was as effective as high and low doses of Dexa in reduction of inflammation (FIG. 26) and decrease of thickness of animal ear (FIG. 27).

### Example 6: Evaluation of pHLIP®-Dexa in Enhancement of Cytotoxic Effect of a pHLIP Conjugated Chemotherapeutic Agent (pHLIP®-Calicheamicin) on Myeloma Cancer Cells

Dexamethasone at high doses can kill myeloma cancer cells, or at low doses, can enhance effect of cytotoxic molecules. pHLIP-Dexa was evaluated for the enhancement of cytotoxic effect using pHLIP-calicheamicin (pHLIP-Cal).

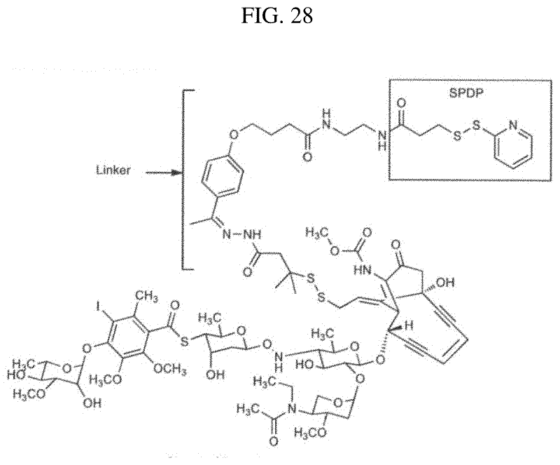

Calicheamicin modified with SPDP was synthesized and purified by Cfm, GmbH (FIG. 28). Calicheamicin-SPDP was used to conjugate with Cys residue of pHLIP® peptide's membrane-inserting end to obtain pHLIP-S-S-calicheamicin. The pHLIP® peptide used in the study: ADDQNPWRAYLDLLFPTDTLLLDLLWCA (SEQ ID NO: 3) was prepared by solid-phase synthesis. Purification of the pHLIP-S-S-calicheamicin is conducted using RP-HPLC (the gradient: 10 mM TEAA buffer and acetonitrile), followed by lyophilization. The construct purity was established by analytical RP-HPLC. Construct concentration was calculated by absorbance at 280 nm for pHLIP® peptide with correction on absorbance of calicheamicin. Myeloma cancer cells were loaded in the wells (30,000 cells/well) and incubated overnight. Increasing amounts of pHLIP-Cal in absence and presence of 5 μM of pHLIP-Dexa were added to the cells in DMEM without FBS for 2 hrs, followed by addition of DMEM with FBS (to have 10% FBS as final concentration in the well). Cell viability was assessed after 22 hours using the colorimetric CellTiter 96 AQᵤₑₒᵤₛ One Solution Cell Proliferation Assay by absorption measurement at 490 nm.

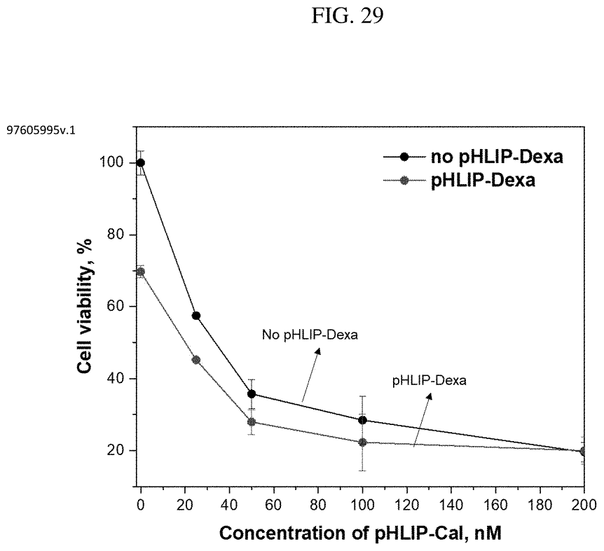

The obtained results indicate that pHLIP-Dexa alone induced myeloma cancer cells death and enhanced cytotoxic effect of pHLIP-Cal (FIG. 29)

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

A small molecule is a compound that is less than 2000 daltons in mass. The molecular mass of the small molecule is preferably less than 1000 daltons, more preferably less than 600 daltons, e.g., the compound is less than 500 daltons, 400 daltons, 300 daltons, 200 daltons, or 100 daltons.

As used herein, an “isolated” or “purified” nucleic acid molecule, polynucleotide, polypeptide, or protein, or chemical compound is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis. A purified or isolated polynucleotide (ribonucleic acid (RNA) or deoxyribonucleic acid (DNA)) or polypeptide is free of the amino acid sequences, or nucleic acid sequences that flank it in its naturally-occurring state. A purified or isolated polynucleotide (ribonucleic acid (RNA) or deoxyribonucleic acid (DNA)) is free of the genes or sequences that flank it in its natural-occurring state. A purified or isolated polypeptide is free of the amino acids or sequences that flank it in its naturally-occurring state. Purified also defines a degree of sterility that is safe for administration to a human subject, e.g., lacking infectious or toxic agents.

Similarly, by “substantially pure” is meant a nucleotide or polypeptide that has been separated from the components that naturally accompany it. Typically, the nucleotides and polypeptides are substantially pure when they are at least 60%, 70%, 80%, 90%, 95%, or even 99%, by weight, free from the proteins and naturally-occurring organic molecules with they are naturally associated.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

The terms “subject,” “patient,” “individual,” and the like as used herein are not intended to be limiting and can be generally interchanged. That is, an individual described as a “patient” does not necessarily have a given disease, but may be merely seeking medical advice.

As used herein, the singular forms “a,” “an,” and “the” include the plural reference unless the context clearly dictates otherwise. Thus, for example, a reference to “a disease,” “a disease state”, or “a nucleic acid” is a reference to one or more such embodiments, and includes equivalents thereof known to those skilled in the art and so forth.

As used herein, “treating” encompasses, e.g., inhibition, regression, or stasis of the progression of a disorder. Treating also encompasses the prevention or amelioration of any symptom or symptoms of the disorder. As used herein, “inhibition” of disease progression or a disease complication in a subject means preventing or reducing the disease progression and/or disease complication in the subject.

As used herein, a “symptom” associated with a disorder includes any clinical or laboratory manifestation associated with the disorder, and is not limited to what the subject can feel or observe.

As used herein, “effective” when referring to an amount of a therapeutic compound refers to the quantity of the compound that is sufficient to yield a desired therapeutic response without undue adverse side effects (such as toxicity, irritation, or allergic response) commensurate with a reasonable benefit/risk ratio when used in the manner of this disclosure.

As used herein, “pharmaceutically acceptable” carrier or excipient refers to a carrier or excipient that is suitable for use with humans and/or animals without undue adverse side effects (such as toxicity, irritation, and allergic response) commensurate with a reasonable benefit/risk ratio. It can be, e.g., a pharmaceutically acceptable solvent, suspending agent or vehicle, for delivering the instant compounds to the subject.

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

“Percentage of sequence identity” is determined by comparing two optimally aligned sequences over a comparison window, wherein the portion of the polynucleotide or polypeptide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “identical” or percent “identity,” in the context of two or more nucleic acids or polypeptide sequences, refer to two or more sequences or subsequences that are the same or have a specified percentage of amino acid residues or nucleotides that are the same (e.g., 50%, 55%, 60%, 65%, 70%, 75%, 80%, 85%, 90%, 91%, 92%, 93%, 94%, 95%, 96%, 97%, 98%, 99%, or more identity over a specified region, e.g., of an entire polypeptide sequence or an individual domain thereof), when compared and aligned for maximum correspondence over a comparison window, or designated region as measured using a sequence comparison algorithm or by manual alignment and visual inspection. Such sequences that are at least about 80% identical are said to be “substantially identical.” In some embodiments, two sequences are 100% identical. In certain embodiments, two sequences are 100% identical over the entire length of one of the sequences (e.g., the shorter of the two sequences where the sequences have different lengths). In various embodiments, identity may refer to the complement of a test sequence. In some embodiments, the identity exists over a region that is at least about 10 to about 100, about 20 to about 75, about 30 to about 50 amino acids or nucleotides in length. In certain embodiments, the identity exists over a region that is at least about 50 amino acids in length, or more preferably over a region that is 100 to 500, 100 to 200, 150 to 200, 175 to 200, 175 to 225, 175 to 250, 200 to 225, 200 to 250 or more amino acids in length.

For sequence comparison, typically one sequence acts as a reference sequence, to which test sequences are compared. In various embodiments, when using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A the “comparison window” refers to a segment of any one of the number of contiguous positions (e.g., least about 10 to about 100, about 20 to about 75, about 30 to about 50, 100 to 500, 100 to 200, 150 to 200, 175 to 200, 175 to 225, 175 to 250, 200 to 225, 200 to 250) in which a sequence may be compared to a reference sequence of the same number of contiguous positions after the two sequences are optimally aligned. In various embodiments, a comparison window is the entire length of one or both of two aligned sequences. In some embodiments, two sequences being compared comprise different lengths, and the comparison window is the entire length of the longer or the shorter of the two sequences. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

In various embodiments, an algorithm that is suitable for determining percent sequence identity and sequence similarity are the BLAST and BLAST 2.0 algorithms, which are described in Altschul et al., Nuc. Acids Res. 25:3389-3402 (1977) and Altschul et al., J. Mol. Biol. 215:403-410 (1990), respectively. BLAST and BLAST 2.0 may be used, with the parameters described herein, to determine percent sequence identity for nucleic acids and proteins. Software for performing BLAST analyses is publicly available through the National Center for Biotechnology Information, as known in the art. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed:

1. A composition comprising a corticosteroid and a pHLIP® peptide.

2. The composition of claim 1, wherein said corticosteroid suppresses inflammation and an immune reaction locally in a diseased tissue.

3. The composition of claim 1, wherein said corticosteroid comprises, dexamethasone, betamethasone, or a derivative thereof.

4. The composition of claim 1, wherein said corticosteroid comprises preferentially targets inflamed tissues.

5. The composition of claim 1, further comprising a linker between said corticosteroid and said pHLIP® peptide.

6. The composition of claim 5, wherein said linker comprises a disulfide bond, an acid-liable bond, or an ester bond.

7. The composition of claim 5, wherein said linker is cleavable.

8. The composition of claim 5, wherein said linker is not cleavable.

9. The composition of claim 8, wherein said linker is a polyethylene glycol (PEG) polymer.

10. The composition of claim 9, wherein said linker is a PEG polymer comprising of 2 to 24 units.

11. The composition of claim 1, further comprising a polar modulator.

12. The composition of claim 1, wherein said pHLIP peptide comprises the sequence ADDQNPWRAYLDLLFPTDTLLLDLLWXA (SEQ ID NO: 1) or ADQDNPWRAYLDLLFPTDTLLLDLLWXA (SEQ ID NO: 2), wherein upper case “X” indicates any amino acid residue.

13. The composition of claim 10, wherein said “X” is lysine (Lys), Cysteine (Cys), or an Azido-containing amino acid.

14. The composition of claim 1, comprising the following structure: A-L-Cs
wherein “A” is a pHLIP® peptide comprising the sequence ADDQNPWRAYLDLLFPTDTLLLDLLWXA (SEQ ID NO: 1), “L” is a polyethylene glycol linker; “Cs” is a corticosteroid, and wherein each “-” is a covalent bond.

15. A method for inducing local immunosuppression, comprising administering to a subject a composition comprising a corticosteroid and a pHLIP® peptide, wherein immunosuppression is locally induced at an anatomical location comprising an acidic pH.

16. The method of claim 14, wherein said subject comprises an inflamed tissue.

17. The method of claim 14, wherein said composition is injected directly into a diseased tissue.

18. The method of claim 14, wherein said composition is topically applied.

19. The method of claim 14, wherein said composition is systemically administered.

20. The method of claim 14, wherein said corticosteroid is delivered into the cytosol of a macrophage.

21. The method of claim 14, wherein said corticosteroid enters a cell of an inflamed tissue to induce a biological effect within said inflamed tissue.

22. The method of claim 14, wherein said corticosteroid is delivered intracellularly to induce a biological effect.

23. The method of claim 14, wherein said composition preferentially targets said corticosteroid to a diseased tissue, thereby minimizing systemic immunosuppression and reducing side effects.

24. The method of reducing inflammation in a subject, comprising administering to the subject a composition comprising a corticosteroid and a pHLIP® peptide.

25. The method of claim 24, wherein said inflammation comprises bleomycin-induced inflammation.

26. The method of claim 24, wherein said inflammation comprises arthritis.

27. The method of claim 24, wherein said inflammation comprises dermatitis.

28. The method of claim 24, wherein the corticosteroid comprises dexamethasone.

29. A method for enhancing the cytotoxicity of a corticosteroid in a subject, comprising administering to the subject a composition comprising a corticosteroid and a pHLIP® peptide.

30. The method of claim 29, wherein the corticosteroid comprises dexamethasone.

31. The method of claim 29, wherein the composition comprising the corticosteroid and the pHLIP® peptide enhances the cytotoxicity of the corticosteroid by 10%, 20% 30%, 40% 50% 75%, 2-fold, 3-fold, 5-fold, 7-fold, 10-fold or more compared to the level of the corticosteroid alone.

32. A method for treating cancer in a subject, comprising administering to the subject a composition comprising (a) a corticosteroid and a pHLIP® peptide, and (b) a chemotherapeutic agent and pHLIP® peptide.


SEQUENCE LISTING (217 sequences)

SEQ ID NO: 1 - protein, 28 aa, Artificial Sequence
  Synthetic petide
  MISC_FEATURE (27)..(27): X is any amino acid
ADDQNPWRAYLDLLFPTDTLLLDLLWXA

SEQ ID NO: 2 - protein, 28 aa, Artificial Sequence
  Synthetic petide
  MISC_FEATURE (27)..(27): X is any amino acid
ADQDNPWRAYLDLLFPTDTLLLDLLWXA

SEQ ID NO: 3 - protein, 28 aa, Artificial Sequence
  Synthetic petide
ADDQNPWRAYLDLLFPTDTLLLDLLWCA

SEQ ID NO: 4 - protein, 35 aa, Artificial Sequence
  Synthetic petide
AEQNPIYWARYADWLFTTPLLLLDLALLVDADEGT

SEQ ID NO: 5 - protein, 7 aa, Artificial Sequence
  Synthetic petide
AEQNPIY

SEQ ID NO: 6 - protein, 22 aa, Artificial Sequence
  Synthetic petide
WARYADWLFTTPLLLLDLALLV

SEQ ID NO: 7 - protein, 6 aa, Artificial Sequence
  Synthetic peptide
DADEGT

SEQ ID NO: 8 - protein, 36 aa, Artificial Sequence
  Description of Artificial Sequence Synthetic polypeptide
GGEQNPIYWARYADWLFTTPLLLLDLALLVDADEGT

SEQ ID NO: 9 - protein, 35 aa, Artificial Sequence; identical to SEQ ID NO: 4
  Description of Artificial Sequence Synthetic polypeptide

SEQ ID NO: 10 - protein, 29 aa, Artificial Sequence
  Description of Artificial Sequence Synthetic peptide
ADDQNPWRAYLDLLFPTDTLLLDLLWCKA

SEQ ID NO: 11 - protein, 32 aa, Artificial Sequence
  Description of Artificial Sequence Synthetic polypeptide
ADDQNPWRAYLDLLFPTDTLLLDLLWDADECG

SEQ ID NO: 12 - protein, 32 aa, Artificial Sequence
  Description of Artificial Sequence Synthetic polypeptide
ADDQNPWRAYLDLLFPTDTLLLDLLWDADEKG

SEQ ID NO: 13 - protein, 33 aa, Artificial Sequence
  Description of Artificial Sequence Synthetic polypeptide
ADDQNPWRAYLDLLFPTDTLLLDLLWDADEKCG

SEQ ID NO: 14 - protein, 32 aa, Artificial Sequence
  Description of Artificial Sequence Synthetic polypeptide
ACDDQNPWRAYLDLLFPTDTLLLDLLWDADEG

SEQ ID NO: 15 - protein, 32 aa, Artificial Sequence
  Description of Artificial Sequence Synthetic polypeptide
AKDDQNPWRAYLDLLFPTDTLLLDLLWDADEG

SEQ ID NO: 16 - protein, 38 aa, Artificial Sequence
  Description of Artificial Sequence Synthetic polypeptide
AAEQNPIYWARYADWLFTTPLLLLDLALLVDADEGTCG

SEQ ID NO: 17 - protein, 36 aa, Artificial Sequence
  Description of Artificial Sequence Synthetic polypeptide
AEQNPIYWARYADWLFTTPLLLLDLALLVDADEGCT

SEQ ID NO: 18 - protein, 38 aa, Artificial Sequence
  Description of Artificial Sequence Synthetic polypeptide
GGEQNPIYWARYADWLFTTPLLLLDLALLVDADEGTCG

SEQ ID NO: 19 - protein, 37 aa, Artificial Sequence
  Description of Artificial Sequence Synthetic polypeptide
ACEQNPIYWARYADWLFTTPLLLLDLALLVDADEGTG

SEQ ID NO: 20 - protein, 36 aa, Artificial Sequence
  Description of Artificial Sequence Synthetic polypeptide
ACEQNPIYWARYADWLFTTPLLLLDLALLVDADEGT

SEQ ID NO: 21 - protein, 36 aa, Artificial Sequence
  Description of Artificial Sequence Synthetic polypeptide
AKEQNPIYWARYADWLFTTPLLLLDLALLVDADEGT

SEQ ID NO: 22 - protein, 37 aa, Artificial Sequence
  Description of Artificial Sequence Synthetic polypeptide
AKEQNPIYWARYADWLFTTPLLLLDLALLVDADEGTG

SEQ ID NO: 23 - protein, 39 aa, Artificial Sequence
  Description of Artificial Sequence Synthetic polypeptide
AAEQNPIYWARYADWLFTTPLLLLDLALLVDADEGTKCG

SEQ ID NO: 24 - protein, 36 aa, Artificial Sequence
  Description of Artificial Sequence Synthetic polypeptide
AKEQNPIYWARYADWLFTTPLLLLDLALLVDADECT

SEQ ID NO: 25 - protein, 37 aa, Artificial Sequence
  Description of Artificial Sequence Synthetic polypeptide
ACEQNPIYWARYANWLFTTPLLLLNLALLVDADEGTG

SEQ ID NO: 26 - protein, 36 aa, Artificial Sequence
  Description of Artificial Sequence Synthetic polypeptide
ACEQNPIYWARYANWLFTTPLLLLNLALLVDADEGT

SEQ ID NO: 27 - protein, 37 aa, Artificial Sequence
  Description of Artificial Sequence Synthetic polypeptide
ACEQNPIYWARYAKWLFTTPLLLLKLALLVDADEGTG

SEQ ID NO: 28 - protein, 36 aa, Artificial Sequence
  Description of Artificial Sequence Synthetic polypeptide
GGEQNPIYWARYADWLFTTPLLLLDLALLVNANQGT

SEQ ID NO: 29 - protein, 36 aa, Artificial Sequence
  Description of Artificial Sequence Synthetic polypeptide
AAEQNPIYWARYADWLFTTPLLLLALALLVDADEGT

SEQ ID NO: 30 - protein, 39 aa, Artificial Sequence
  Description of Artificial Sequence Synthetic polypeptide
AAEQNPIYWARYADWLFTTPLLLLELALLVDADEGTKCG

SEQ ID NO: 31 - protein, 36 aa, Artificial Sequence
  Description of Artificial Sequence Synthetic polypeptide
AAEQNPIYWARYAAWLFTTPLLLLDLALLVDADEGT

SEQ ID NO: 32 - protein, 36 aa, Artificial Sequence
  Description of Artificial Sequence Synthetic polypeptide
AAEQNPIYWARYADWLFTTALLLLDLALLVDADEGT

SEQ ID NO: 33 - protein, 36 aa, Artificial Sequence
  Description of Artificial Sequence Synthetic polypeptide
AAEQNPIYWARYADWLFTTPLLLLELALLVDADEGT

SEQ ID NO: 34 - protein, 36 aa, Artificial Sequence
  Description of Artificial Sequence Synthetic polypeptide
AAEQNPIYWARYAEWLFTTPLLLLDLALLVDADEGT

SEQ ID NO: 35 - protein, 39 aa, Artificial Sequence
  Description of Artificial Sequence Synthetic polypeptide
AAEQNPIIYWARYADWLFTDLPLLLLDLLALLVDADEGT

SEQ ID NO: 36 - protein, 37 aa, Artificial Sequence
  Description of Artificial Sequence Synthetic polypeptide
GEQNPIYWAQYADWLFTTPLLLLDLALLVDADEGTCG

SEQ ID NO: 37 - protein, 38 aa, Artificial Sequence
  Description of Artificial Sequence Synthetic polypeptide
GGEQNPIYWARYADWLFTTPLLLDLLALLVDADEGTCG

SEQ ID NO: 38 - protein, 38 aa, Artificial Sequence
  Description of Artificial Sequence Synthetic polypeptide
GGEQNPIYWARYADWLFTTPLLLLLDALLVDADEGTCG

SEQ ID NO: 39 - protein, 38 aa, Artificial Sequence
  Description of Artificial Sequence Synthetic polypeptide
GGEQNPIYWARYDAWLFTTPLLLLDLALLVDADEGTCG

SEQ ID NO: 40 - protein, 38 aa, Artificial Sequence
  Description of Artificial Sequence Synthetic polypeptide
GGEQNPIYWARYAWDLFTTPLLLLDLALLVDADEGTCG

SEQ ID NO: 41 - protein, 36 aa, Artificial Sequence
  Description of Artificial Sequence Synthetic polypeptide
AAEQNPIYWARYADWLFTTGLLLLDLALLVDADEGT

SEQ ID NO: 42 - protein, 37 aa, Artificial Sequence
  Description of Artificial Sequence Synthetic polypeptide
DDDEDNPIYWARYADWLFTTPLLLLHGALLVDADECT

SEQ ID NO: 43 - protein, 36 aa, Artificial Sequence
  Description of Artificial Sequence Synthetic polypeptide
DDDEDNPIYWARYADWLFTTPLLLLHGALLVDADET

SEQ ID NO: 44 - protein, 38 aa, Artificial Sequence
  Description of Artificial Sequence Synthetic polypeptide
DDDEDNPIYWARYAHWLFTTPLLLLHGALLVDADEGCT

SEQ ID NO: 45 - protein, 37 aa, Artificial Sequence
  Description of Artificial Sequence Synthetic polypeptide
CDDDEDNPIYWARYAHWLFTTPLLLLHGALLVDADET

SEQ ID NO: 46 - protein, 37 aa, Artificial Sequence
  Description of Artificial Sequence Synthetic polypeptide
DDDEDNPIYWARYAHWLFTTPLLLLHGALLVDADEGT

SEQ ID NO: 47 - protein, 37 aa, Artificial Sequence
  Description of Artificial Sequence Synthetic polypeptide
DDDEDNPIYWARYAHWLFTTPLLLLHGALLVNADECT

SEQ ID NO: 48 - protein, 37 aa, Artificial Sequence
  Description of Artificial Sequence Synthetic polypeptide
DDDEDNPIYWARYAHWLFTTPLLLLHGALLVNADEGT

SEQ ID NO: 49 - protein, 37 aa, Artificial Sequence
  Description of Artificial Sequence Synthetic polypeptide
DDDEDNPIYWARYAHWLFTTPLLLLHGALLVNANECT

SEQ ID NO: 50 - protein, 37 aa, Artificial Sequence
  Description of Artificial Sequence Synthetic polypeptide
DDDEDNPIYWARYAHWLFTTPLLLLHGALLVNANEGT

SEQ ID NO: 51 - protein, 34 aa, Artificial Sequence
  Description of Artificial Sequence Synthetic polypeptide
AEQNPIYWARYADFLFTTPLLLLDLALLVDADET

SEQ ID NO: 52 - protein, 35 aa, Artificial Sequence
  Description of Artificial Sequence Synthetic polypeptide
AEQNPIYFARYADWLFTTPLLLLDLALLVDADEGT

SEQ ID NO: 53 - protein, 34 aa, Artificial Sequence
  Description of Artificial Sequence Synthetic polypeptide
AEQNPIYFARYADFLFTTPLLLLDLALLWDADET

SEQ ID NO: 54 - protein, 32 aa, Artificial Sequence
  Description of Artificial Sequence Synthetic polypeptide
AKEDQNPYWARYADWLFTTPLLLLDLALLVDG

SEQ ID NO: 55 - protein, 32 aa, Artificial Sequence
  Description of Artificial Sequence Synthetic polypeptide
ACEDQNPYWARYADWLFTTPLLLLDLALLVDG

SEQ ID NO: 56 - protein, 32 aa, Artificial Sequence
  Description of Artificial Sequence Synthetic polypeptide
AEDQNPYWARYADWLFTTPLLLLDLALLVDCG

SEQ ID NO: 57 - protein, 32 aa, Artificial Sequence
  Description of Artificial Sequence Synthetic polypeptide
AEDQNPYWARYADWLFTTPLLLLELALLVECG

SEQ ID NO: 58 - protein, 32 aa, Artificial Sequence
  Description of Artificial Sequence Synthetic polypeptide
AKEDQNDPYWARYADWLFTTPLLLLDLALLVG

SEQ ID NO: 59 - protein, 30 aa, Artificial Sequence
  Description of Artificial Sequence Synthetic polypeptide
AKEDQNPYWRAYADLFTPLTLLDLLALWDG

SEQ ID NO: 60 - protein, 31 aa, Artificial Sequence
  Description of Artificial Sequence Synthetic polypeptide
AEDQNPYWARYADWLFTTPLLLLELALLVCG

SEQ ID NO: 61 - protein, 30 aa, Artificial Sequence
  Description of Artificial Sequence Synthetic polypeptide
ACEDQNPYWRAYADLFTPLTLLDLLALWDG

SEQ ID NO: 62 - protein, 27 aa, Artificial Sequence
  Description of Artificial Sequence Synthetic peptide
ACDDQNPWRAYLDLLFPTDTLLLDLLW

SEQ ID NO: 63 - protein, 28 aa, Artificial Sequence
  Description of Artificial Sequence Synthetic peptide
AKDDQNPWRAYLDLLFPTDTLLLDLLWC

SEQ ID NO: 64 - protein, 27 aa, Artificial Sequence
  Description of Artificial Sequence Synthetic peptide
ACEEQNPWRAYLELLFPTETLLLELLW

SEQ ID NO: 65 - protein, 25 aa, Artificial Sequence
  Description of Artificial Sequence Synthetic peptide
ACDDQNPWARYLDWLFPTDTLLLDL

SEQ ID NO: 66 - protein, 24 aa, Artificial Sequence
  Description of Artificial Sequence Synthetic peptide
CDNNNPWRAYLDLLFPTDTLLLDW

SEQ ID NO: 67 - protein, 24 aa, Artificial Sequence
  Description of Artificial Sequence Synthetic peptide
CEEQQPWAQYLELLFPTETLLLEW

SEQ ID NO: 68 - protein, 24 aa, Artificial Sequence
  Description of Artificial Sequence Synthetic peptide
CEEQQPWRAYLELLFPTETLLLEW

SEQ ID NO: 69 - protein, 37 aa, Artificial Sequence
  Description of Artificial Sequence Synthetic polypeptide
CDDDDDNPNYWARYANWLFTTPLLLLNGALLVEAEET

SEQ ID NO: 70 - protein, 36 aa, Artificial Sequence
  Description of Artificial Sequence Synthetic polypeptide
CDDDDDNPNYWARYAPWLFTTPLLLLPGALLVEAEE

SEQ ID NO: 71 - protein, 34 aa, Artificial Sequence
  Description of Artificial Sequence Synthetic polypeptide
TEDADVLLALDLLLLPTTFLWDAYRAWYPNQECA

SEQ ID NO: 72 - protein, 20 aa, Artificial Sequence
  Description of Artificial Sequence Synthetic peptide
AEQNPIYWARYADWLFTTPL

SEQ ID NO: 73 - protein, 21 aa, Artificial Sequence
  Description of Artificial Sequence Synthetic peptide
AEQNPIYWARYADWLFTTPCL

SEQ ID NO: 74 - protein, 21 aa, Artificial Sequence
  Description of Artificial Sequence Synthetic peptide
ACEQNPIYWARYADWLFTTPL

SEQ ID NO: 75 - protein, 20 aa, Artificial Sequence
  Description of Artificial Sequence Synthetic peptide
AEQNPIYFARYADWLFTTPL

SEQ ID NO: 76 - protein, 22 aa, Artificial Sequence
  Description of Artificial Sequence Synthetic peptide
AEQNPIYFARYADLLFPTTLAW

SEQ ID NO: 77 - protein, 22 aa, Artificial Sequence
  Description of Artificial Sequence Synthetic peptide
AEQNPIYWARYADLLFPTTLAF

SEQ ID NO: 78 - protein, 22 aa, Artificial Sequence
  Description of Artificial Sequence Synthetic peptide
AEQNPIYWARYADLLFPTTLAW

SEQ ID NO: 79 - protein, 21 aa, Artificial Sequence
  Description of Artificial Sequence Synthetic peptide
KEDQNPWARYADLLFPTTLAW

SEQ ID NO: 80 - protein, 20 aa, Artificial Sequence
  Description of Artificial Sequence Synthetic peptide
KEDQNPWARYADLLFPTTLW

SEQ ID NO: 81 - protein, 22 aa, Artificial Sequence
  Description of Artificial Sequence Synthetic peptide
ACEDQNPWARYADLLFPTTLAW

SEQ ID NO: 82 - protein, 24 aa, Artificial Sequence
  Description of Artificial Sequence Synthetic peptide
ACEDQNPWARYADWLFPTTLLLLD

SEQ ID NO: 83 - protein, 22 aa, Artificial Sequence
  Description of Artificial Sequence Synthetic peptide
ACEEQNPWARYAELLFPTTLAW

SEQ ID NO: 84 - protein, 24 aa, Artificial Sequence
  Description of Artificial Sequence Synthetic peptide
ACEEQNPWARYAEWLFPTTLLLLE

SEQ ID NO: 85 - protein, 25 aa, Artificial Sequence
  Description of Artificial Sequence Synthetic peptide
ACEEQNPWARYLEWLFPTETLLLEL

SEQ ID NO: 86 - protein, 24 aa, Artificial Sequence
  Description of Artificial Sequence Synthetic peptide
ACEEQNPQAEYAEWLFPTTLLLLE

SEQ ID NO: 87 - protein, 21 aa, Artificial Sequence
  Description of Artificial Sequence Synthetic peptide
  PEPTIDE (1)..(21)
WARYADWLFTTPLLLLDLALL

SEQ ID NO: 88 - protein, 21 aa, Artificial Sequence
  Description of Artificial Sequence Synthetic peptide
  PEPTIDE (1)..(21)
YARYADWLFTTPLLLLDLALL

SEQ ID NO: 89 - protein, 21 aa, Artificial Sequence
  Description of Artificial Sequence Synthetic peptide
  PEPTIDE (1)..(21)
WARYSDWLFTTPLLLYDLGLL

SEQ ID NO: 90 - protein, 21 aa, Artificial Sequence
  Description of Artificial Sequence Synthetic peptide
  PEPTIDE (1)..(21)
WARYTDWFTTPLLLYDLALLA

SEQ ID NO: 91 - protein, 21 aa, Artificial Sequence
  Description of Artificial Sequence Synthetic peptide
  PEPTIDE (1)..(21): See specification as filed for detailed description of substitutions and preferred embodiments
WARYTDWLFTTPLLLYDLGLL

SEQ ID NO: 92 - protein, 21 aa, Artificial Sequence
  Description of Artificial Sequence Synthetic peptide
  peptide (1)..(21): See specification as filed for detailed description of substitutions and preferred embodiments
WARYADWLFTTPLLLLDLSLL

SEQ ID NO: 93 - protein, 21 aa, Artificial Sequence
  Description of Artificial Sequence Synthetic peptide
  Peptide (1)..(21): See specification as filed for detailed description of substitutions and preferred embodiments
LLALDLLLLPTTFLWDAYRAW

SEQ ID NO: 94 - protein, 21 aa, Artificial Sequence
  Description of Artificial Sequence Synthetic peptide
  Peptide (1)..(21): See specification as filed for detailed description of substitutions and preferred embodiments
LLALDLLLLPTTFLWDAYRAY

SEQ ID NO: 95 - protein, 21 aa, Artificial Sequence
  Description of Artificial Sequence Synthetic peptide
  Peptide (1)..(21): See specification as filed for detailed description of substitutions and preferred embodiments
LLGLDYLLLPTTFLWDSYRAW

SEQ ID NO: 96 - protein, 21 aa, Artificial Sequence
  Description of Artificial Sequence Synthetic peptide
  Peptide (1)..(21): See specification as filed for detailed description of substitutions and preferred embodiments
ALLALDYLLLPTTFWDTYRAW

SEQ ID NO: 97 - protein, 21 aa, Artificial Sequence
  Description of Artificial Sequence Synthetic peptide
  Peptide (1)..(21): See specification as filed for detailed description of substitutions and preferred embodiments
LLGLDYLLLPTTFLWDTYRAW

SEQ ID NO: 98 - protein, 21 aa, Artificial Sequence
  Description of Artificial Sequence Synthetic peptide
  peptide (1)..(21): See specification as filed for detailed description of substitutions and preferred embodiments
LLSLDLLLLPTTFLWDAYRAW

SEQ ID NO: 99 - protein, 26 aa, Artificial Sequence
  Description of Artificial Sequence Synthetic peptide
  peptide (1)..(21): See specification as filed for detailed description of substitutions and preferred embodiments
GLAGLLGLEGLLGLPLGLLEGLWLGL

SEQ ID NO: 100 - protein, 26 aa, Artificial Sequence
  Description of Artificial Sequence Synthetic peptide
  peptide (1)..(26): See specification as filed for detailed description of substitutions and preferred embodiments
LGLWLGELLGLPLGLLGELGLLGALG

SEQ ID NO: 101 - protein, 20 aa, Artificial Sequence
  Description of Artificial Sequence Synthetic peptide
  peptide (1)..(20): See specification as filed for detailed description of substitutions and preferred embodiments
WRAYLDLLFPTDTLLLDLLW

SEQ ID NO: 102 - protein, 20 aa, Artificial Sequence
  Description of Artificial Sequence Synthetic peptide
  peptide (1)..(20): See specification as filed for detailed description of substitutions and preferred embodiments
WLLDLLLTDTPFLLDLYARW

SEQ ID NO: 103 - protein, 18 aa, Artificial Sequence
  Description of Artificial Sequence Synthetic peptide
  peptide (1)..(18): See specification as filed for detailed description of substitutions and preferred embodiments
WARYLEWLFPTETLLLEL

SEQ ID NO: 104 - protein, 18 aa, Artificial Sequence
  Description of Artificial Sequence Synthetic peptide
  peptide (1)..(18): See specification as filed for detailed description of substitutions and preferred embodiments
WAQYLELLFPTETLLLEW

SEQ ID NO: 105 - protein, 18 aa, Artificial Sequence
  Description of Artificial Sequence Synthetic peptide
  peptide (1)..(18): See specification as filed for detailed description of substitutions and preferred embodiments
LELLLTETPFLWELYRAW

SEQ ID NO: 106 - protein, 18 aa, Artificial Sequence
  Description of Artificial Sequence Synthetic peptide
  peptide (1)..(18): See specification as filed for detailed description of substitutions and preferred embodiments
WELLLTETPFLLELYQAW

SEQ ID NO: 107 - protein, 17 aa, Artificial Sequence
  Description of Artificial Sequence Synthetic peptide
  peptide (1)..(17): See specification as filed for detailed description of substitutions and preferred embodiments
WLFTTPLLLLNGALLVE

SEQ ID NO: 108 - protein, 17 aa, Artificial Sequence
  Description of Artificial Sequence Synthetic peptide
  Peptide (1)..(17): See specification as filed for detailed description of substitutions and preferred embodiments
WLFTTPLLLLPGALLVE

SEQ ID NO: 109 - protein, 15 aa, Artificial Sequence
  Description of Artificial Sequence Synthetic peptide
  Peptide (1)..(15): See specification as filed for detailed description of substitutions and preferred embodiments
WARYADLLFPTTLAW

SEQ ID NO: 110 - protein, 17 aa, Artificial Sequence
  Description of Artificial Sequence Synthetic peptide
  Peptide (1)..(17): See specification as filed for detailed description of substitutions and preferred embodiments
EVLLAGNLLLLPTTFLW

SEQ ID NO: 111 - protein, 17 aa, Artificial Sequence
  Description of Artificial Sequence Synthetic peptide
  Peptide (1)..(17): See specification as filed for detailed description of substitutions and preferred embodiments
EVLLAGPLLLLPTTFLW

SEQ ID NO: 112 - protein, 15 aa, Artificial Sequence
  Description of Artificial Sequence Synthetic peptide
  Peptide (1)..(15): See specification as filed for detailed description of substitutions and preferred embodiments
WALTTPFLLDAYRAW

SEQ ID NO: 113 - protein, 24 aa, Artificial Sequence
  Description of Artificial Sequence Synthetic peptide
  Peptide (1)..(24): See specification as filed for detailed description of substitutions and preferred embodiments
NLEGFFATLGGEIALWSLVVLAIE

SEQ ID NO: 114 - protein, 22 aa, Artificial Sequence
  Description of Artificial Sequence Synthetic peptide
  Peptide (1)..(22): See specification as filed for detailed description of substitutions and preferred embodiments
EGFFATLGGEIALWSDVVLAIE

SEQ ID NO: 115 - protein, 22 aa, Artificial Sequence
  Description of Artificial Sequence Synthetic peptide
  Peptide (1)..(22): See specification as filed for detailed description of substitutions and preferred embodiments
EGFFATLGGEIPLWSDVVLAIE

SEQ ID NO: 116 - protein, 24 aa, Artificial Sequence
  Description of Artificial Sequence Synthetic peptide
  Peptide (1)..(24): See specification as filed for detailed description of substitutions and preferred embodiments
EIALVVLSWLAIEGGLTAFFGELN

SEQ ID NO: 117 - protein, 22 aa, Artificial Sequence
  Description of Artificial Sequence Synthetic peptide
  Peptide (1)..(22): See specification as filed for detailed description of substitutions and preferred embodiments
EIALVVDSWLAIEGGLTAFFGE

SEQ ID NO: 118 - protein, 22 aa, Artificial Sequence
  Description of Artificial Sequence Synthetic peptide
  Peptide (1)..(22): See specification as filed for detailed description of substitutions and preferred embodiments
EIALVVDSWLPIEGGLTAFFGE

SEQ ID NO: 119 - protein, 21 aa, Artificial Sequence
  Description of Artificial Sequence Synthetic peptide
  Peptide (1)..(21): See specification as filed for detailed description of substitutions and preferred embodiments
ILDLVFGLLFAVTSVDFLVQW

SEQ ID NO: 120 - protein, 21 aa, Artificial Sequence
  Description of Artificial Sequence Synthetic peptide
  Peptide (1)..(21): See specification as filed for detailed description of substitutions and preferred embodiments
WQVLFDVSTVAFLLGFVLDLI

SEQ ID NO: 121 - protein, 34 aa, Artificial Sequence
  Description of Artificial Sequence Synthetic polypeptide
AEQNPIYWARYADWLFTTPLLLLDLALLVDADET

SEQ ID NO: 122 - protein, 34 aa, Artificial Sequence
  Description of Artificial Sequence Synthetic polypeptide
AEQNPIYWARYAEWLFTTPLLLLELALLVEAEET

SEQ ID NO: 123 - protein, 33 aa, Artificial Sequence
  Description of Artificial Sequence Synthetic polypeptide
ADDQNPWRAYLDLLFPDTTDLLLLDLLWDADET

SEQ ID NO: 124 - protein, 33 aa, Artificial Sequence
  Description of Artificial Sequence Synthetic polypeptide
AEEQNPWRAYLELLFPETTELLLLELLWEAEET

SEQ ID NO: 125 - protein, 34 aa, Artificial Sequence
  Description of Artificial Sequence Synthetic polypeptide
  MOD_RES (13)..(13): Gla
AEQNPIYWARYAXWLFTTPLLLLDLALLVDADET

SEQ ID NO: 126 - protein, 34 aa, Artificial Sequence
  Description of Artificial Sequence Synthetic polypeptide
  MOD_RES (24)..(24): Gla
AEQNPIYWARYADWLFTTPLLLLXLALLVDADET

SEQ ID NO: 127 - protein, 34 aa, Artificial Sequence
  Description of Artificial Sequence Synthetic polypeptide
  MOD_RES (13)..(13): Gla
  MOD_RES (24)..(24): Gla
AEQNPIYWARYAXWLFTTPLLLLXLALLVDADET

SEQ ID NO: 128 - protein, 34 aa, Artificial Sequence; identical to SEQ ID NO: 125
  Description of Artificial Sequence Synthetic polypeptide
  MOD_RES (13)..(13): Aad

SEQ ID NO: 129 - protein, 34 aa, Artificial Sequence; identical to SEQ ID NO: 126
  Description of Artificial Sequence Synthetic polypeptide
  MOD_RES (24)..(24): Aad

SEQ ID NO: 130 - protein, 34 aa, Artificial Sequence; identical to SEQ ID NO: 127
  Description of Artificial Sequence Synthetic polypeptide
  MOD_RES (13)..(13): Aad
  MOD_RES (24)..(24): Aad

SEQ ID NO: 131 - protein, 34 aa, Artificial Sequence; identical to SEQ ID NO: 127
  Description of Artificial Sequence Synthetic polypeptide
  MOD_RES (13)..(13): Gla
  MOD_RES (24)..(24): Aad

SEQ ID NO: 132 - protein, 34 aa, Artificial Sequence; identical to SEQ ID NO: 127
  Description of Artificial Sequence Synthetic polypeptide
  MOD_RES (13)..(13): Aad
  MOD_RES (24)..(24): Gla

SEQ ID NO: 133 - protein, 36 aa, Artificial Sequence; identical to SEQ ID NO: 8
  Description of Artificial Sequence Synthetic polypeptide

SEQ ID NO: 134 - protein, 36 aa, Artificial Sequence; identical to SEQ ID NO: 8
  Description of Artificial Sequence Synthetic polypeptide

SEQ ID NO: 135 - protein, 36 aa, Artificial Sequence
  Description of Artificial Sequence Synthetic polypeptide
AAEQNPIYWARYADWLFTTPLLLLDLALLVDADEGT

SEQ ID NO: 136 - protein, 35 aa, Artificial Sequence; identical to SEQ ID NO: 4
  Description of Artificial Sequence Synthetic polypeptide

SEQ ID NO: 137 - protein, 35 aa, Artificial Sequence
  Description of Artificial Sequence Synthetic polypeptide
AEQNPIYWARYANWLFTTPLLLLNLALLVDADEGT

SEQ ID NO: 138 - protein, 35 aa, Artificial Sequence
  Description of Artificial Sequence Synthetic polypeptide
AEQNPIYWARYAKWLFTTPLLLLKLALLVDADEGT

SEQ ID NO: 139 - protein, 36 aa, Artificial Sequence; identical to SEQ ID NO: 28
  Description of Artificial Sequence Synthetic polypeptide

SEQ ID NO: 140 - protein, 36 aa, Artificial Sequence; identical to SEQ ID NO: 29
  Description of Artificial Sequence Synthetic polypeptide

SEQ ID NO: 141 - protein, 36 aa, Artificial Sequence; identical to SEQ ID NO: 31
  Description of Artificial Sequence Synthetic polypeptide

SEQ ID NO: 142 - protein, 36 aa, Artificial Sequence; identical to SEQ ID NO: 32
  Description of Artificial Sequence Synthetic polypeptide

SEQ ID NO: 143 - protein, 36 aa, Artificial Sequence; identical to SEQ ID NO: 33
  Description of Artificial Sequence Synthetic polypeptide

SEQ ID NO: 144 - protein, 36 aa, Artificial Sequence; identical to SEQ ID NO: 34
  Description of Artificial Sequence Synthetic polypeptide

SEQ ID NO: 145 - protein, 39 aa, Artificial Sequence; identical to SEQ ID NO: 35
  Description of Artificial Sequence Synthetic polypeptide

SEQ ID NO: 146 - protein, 34 aa, Artificial Sequence
  Description of Artificial Sequence Synthetic polypeptide
GEQNPIYWAQYADWLFTTPLLLLDLALLVDADEG

SEQ ID NO: 147 - protein, 35 aa, Artificial Sequence
  Description of Artificial Sequence Synthetic polypeptide
GGEQNPIYWARYADWLFTTPLLLDLLALLVDADEG

SEQ ID NO: 148 - protein, 35 aa, Artificial Sequence
  Description of Artificial Sequence Synthetic polypeptide
GGEQNPIYWARYADWLFTTPLLLLLDALLVDADEG

SEQ ID NO: 149 - protein, 36 aa, Artificial Sequence
  Description of Artificial Sequence Synthetic polypeptide
GGEQNPIYWARYDAWLFTTPLLLLDLALLVDADEGT

SEQ ID NO: 150 - protein, 35 aa, Artificial Sequence
  Description of Artificial Sequence Synthetic polypeptide
GGEQNPIYWARYAWDLFTTPLLLLDLALLVDADEG

SEQ ID NO: 151 - protein, 36 aa, Artificial Sequence; identical to SEQ ID NO: 41
  Description of Artificial Sequence Synthetic polypeptide

SEQ ID NO: 152 - protein, 34 aa, Artificial Sequence
  Description of Artificial Sequence Synthetic polypeptide
DDDEDNPIYWARYADWLFTTPLLLLHGALLVDAD

SEQ ID NO: 153 - protein, 35 aa, Artificial Sequence
  Description of Artificial Sequence Synthetic polypeptide
DDDEDNPIYWARYAHWLFTTPLLLLHGALLVDADE

SEQ ID NO: 154 - protein, 33 aa, Artificial Sequence
  Description of Artificial Sequence Synthetic polypeptide
CEQNPIYWARYADWHFTTPLLLLDLALLVDADE

SEQ ID NO: 155 - protein, 34 aa, Artificial Sequence; identical to SEQ ID NO: 51
  Description of Artificial Sequence Synthetic polypeptide

SEQ ID NO: 156 - protein, 33 aa, Artificial Sequence
  Description of Artificial Sequence Synthetic polypeptide
AEQNPIYFARYADWLFTTPLLLLDLALLVDADE

SEQ ID NO: 157 - protein, 34 aa, Artificial Sequence; identical to SEQ ID NO: 53
  Description of Artificial Sequence Synthetic polypeptide

SEQ ID NO: 158 - protein, 32 aa, Artificial Sequence
  Description of Artificial Sequence Synthetic polypeptide
ADNNPWIYARYADLTTFPLLLLDLALLVDFDD

SEQ ID NO: 159 - protein, 32 aa, Artificial Sequence
  Description of Artificial Sequence Synthetic polypeptide
ADNNPFIYARYADLTTWPLLLLDLALLVDFDD

SEQ ID NO: 160 - protein, 32 aa, Artificial Sequence
  Description of Artificial Sequence Synthetic polypeptide
ADNNPFIYARYADLTTFPLLLLDLALLVDWDD

SEQ ID NO: 161 - protein, 32 aa, Artificial Sequence
  Description of Artificial Sequence Synthetic polypeptide
ADNNPFPYARYADLTTWILLLLDLALLVDFDD

SEQ ID NO: 162 - protein, 32 aa, Artificial Sequence
  Description of Artificial Sequence Synthetic polypeptide
ADNNPFIYAYRADLTTFPLLLLDLALLVDWDD

SEQ ID NO: 163 - protein, 32 aa, Artificial Sequence
  Description of Artificial Sequence Synthetic polypeptide
ADNNPFIYATYADLRTFPLLLLDLALLVDWDD

SEQ ID NO: 164 - protein, 33 aa, Artificial Sequence
  Description of Artificial Sequence Synthetic polypeptide
TEDADVLLALDLLLLPTTFLWDAYRAWYPNQEA

SEQ ID NO: 165 - protein, 30 aa, Artificial Sequence
  Description of Artificial Sequence Synthetic polypeptide
AEDQNPYWARYADWLFTTPLLLLDLALLVD

SEQ ID NO: 166 - protein, 30 aa, Artificial Sequence
  Description of Artificial Sequence Synthetic polypeptide
AEDQNPYWARYADWLFTTPLLLLELALLVE

SEQ ID NO: 167 - protein, 28 aa, Artificial Sequence
  Description of Artificial Sequence Synthetic peptide
AEDQNPYWRAYADLFTPLTLLDLLALWD

SEQ ID NO: 168 - protein, 26 aa, Artificial Sequence
  Description of Artificial Sequence Synthetic peptide
ADDQNPWRAYLDLLFPTDTLLLDLLW

SEQ ID NO: 169 - protein, 30 aa, Artificial Sequence
  Description of Artificial Sequence Synthetic polypeptide
ADDQNPWRAYLDLLFPTDTLLLDLLWDADE

SEQ ID NO: 170 - protein, 26 aa, Artificial Sequence
  Description of Artificial Sequence Synthetic peptide
  MOD_RES (12)..(12): Gla
ADDQNPWRAYLXLLFPTDTLLLDLLW

SEQ ID NO: 171 - protein, 26 aa, Artificial Sequence
  Description of Artificial Sequence Synthetic peptide
  MOD_RES (18)..(18): Gla
ADDQNPWRAYLDLLFPTXTLLLDLLW

SEQ ID NO: 172 - protein, 26 aa, Artificial Sequence
  Description of Artificial Sequence Synthetic peptide
  MOD_RES (23)..(23): Gla
ADDQNPWRAYLDLLFPTDTLLLXLLW

SEQ ID NO: 173 - protein, 26 aa, Artificial Sequence
  Description of Artificial Sequence Synthetic peptide
  MOD_RES (12)..(12): Gla
  MOD_RES (18)..(18): Gla
ADDQNPWRAYLXLLFPTXTLLLDLLW

SEQ ID NO: 174 - protein, 26 aa, Artificial Sequence
  Description of Artificial Sequence Synthetic peptide
  MOD_RES (12)..(12): Gla
  MOD_RES (23)..(23): Gla
ADDQNPWRAYLXLLFPTDTLLLXLLW

SEQ ID NO: 175 - protein, 26 aa, Artificial Sequence
  Description of Artificial Sequence Synthetic peptide
  MOD_RES (18)..(18): Gla
  MOD_RES (23)..(23): Gla
ADDQNPWRAYLDLLFPTXTLLLXLLW

SEQ ID NO: 176 - protein, 26 aa, Artificial Sequence
  Description of Artificial Sequence Synthetic peptide
  MOD_RES (12)..(12): Gla
  MOD_RES (18)..(18): Gla
  MOD_RES (23)..(23): Gla
ADDQNPWRAYLXLLFPTXTLLLXLLW

SEQ ID NO: 177 - protein, 26 aa, Artificial Sequence
  Description of Artificial Sequence Synthetic peptide
  MOD_RES (12)..(12): Aad
ADDQNPWRAYLXLLFPTDTLLLDLLW

SEQ ID NO: 178 - protein, 26 aa, Artificial Sequence
  Description of Artificial Sequence Synthetic peptide
  MOD_RES (18)..(18): Aad
ADDQNPWRAYLDLLFPTXTLLLDLLW

SEQ ID NO: 179 - protein, 26 aa, Artificial Sequence
  Description of Artificial Sequence Synthetic peptide
  MOD_RES (23)..(23): Aad
ADDQNPWRAYLDLLFPTDTLLLXLLW

SEQ ID NO: 180 - protein, 26 aa, Artificial Sequence
  Description of Artificial Sequence Synthetic peptide
  MOD_RES (12)..(12): Aad
  MOD_RES (18)..(18): Aad
ADDQNPWRAYLXLLFPTXTLLLDLLW

SEQ ID NO: 181 - protein, 26 aa, Artificial Sequence
  Description of Artificial Sequence Synthetic peptide
  MOD_RES (12)..(12): Aad
  MOD_RES (23)..(23): Aad
ADDQNPWRAYLXLLFPTDTLLLXLLW

SEQ ID NO: 182 - protein, 26 aa, Artificial Sequence
  Description of Artificial Sequence Synthetic peptide
  MOD_RES (18)..(18): Aad
  MOD_RES (23)..(23): Aad
ADDQNPWRAYLDLLFPTXTLLLXLLW

SEQ ID NO: 183 - protein, 26 aa, Artificial Sequence
  Description of Artificial Sequence Synthetic peptide
  MOD_RES (12)..(12): Aad
  MOD_RES (18)..(18): Aad
  MOD_RES (23)..(23): Aad
ADDQNPWRAYLXLLFPTXTLLLXLLW

SEQ ID NO: 184 - protein, 26 aa, Artificial Sequence
  Description of Artificial Sequence Synthetic peptide
  MOD_RES (12)..(12): Gla
  MOD_RES (18)..(18): Aad
ADDQNPWRAYLXLLFPTXTLLLDLLW

SEQ ID NO: 185 - protein, 26 aa, Artificial Sequence
  Description of Artificial Sequence Synthetic peptide
  MOD_RES (12)..(12): Gla
  MOD_RES (23)..(23): Aad
ADDQNPWRAYLXLLFPTDTLLLXLLW

SEQ ID NO: 186 - protein, 26 aa, Artificial Sequence
  Description of Artificial Sequence Synthetic peptide
  MOD_RES (12)..(12): Gla
  MOD_RES (18)..(18): Gla
  MOD_RES (23)..(23): Aad
ADDQNPWRAYLXLLFPTXTLLLXLLW

SEQ ID NO: 187 - protein, 26 aa, Artificial Sequence
  Description of Artificial Sequence Synthetic peptide
  MOD_RES (12)..(12): Aad
  MOD_RES (18)..(18): Gla
ADDQNPWRAYLXLLFPTXTLLLDLLW

SEQ ID NO: 188 - protein, 26 aa, Artificial Sequence
  Description of Artificial Sequence Synthetic peptide
  MOD_RES (12)..(12): Aad
  MOD_RES (23)..(23): Gla
ADDQNPWRAYLXLLFPTDTLLLXLLW

SEQ ID NO: 189 - protein, 26 aa, Artificial Sequence
  Description of Artificial Sequence Synthetic peptide
  MOD_RES (12)..(12): Gla
  MOD_RES (18)..(18): Aad
  MOD_RES (23)..(23): Gla
ADDQNPWRAYLXLLFPTXTLLLXLLW

SEQ ID NO: 190 - protein, 29 aa, Artificial Sequence
  Description of Artificial Sequence Synthetic peptide
GEEQNPWLGAYLDLLFPLELLGLLELGLW

SEQ ID NO: 191 - protein, 28 aa, Artificial Sequence
  Description of Artificial Sequence Synthetic peptide
ADDDDDDPWQAYLDLLFPTDTLLLDLLW

SEQ ID NO: 192 - protein, 26 aa, Artificial Sequence
  Description of Artificial Sequence Synthetic peptide
AEEQNPWRAYLELLFPTETLLLELLW

SEQ ID NO: 193 - protein, 24 aa, Artificial Sequence
  Description of Artificial Sequence Synthetic peptide
ADDQNPWARYLDWLFPTDTLLLDL

SEQ ID NO: 194 - protein, 23 aa, Artificial Sequence
  Description of Artificial Sequence Synthetic peptide
DNNNPWRAYLDLLFPTDTLLLDW

SEQ ID NO: 195 - protein, 24 aa, Artificial Sequence
  Description of Artificial Sequence Synthetic peptide
AEEQNPWARYLEWLFPTETLLLEL

SEQ ID NO: 196 - protein, 25 aa, Artificial Sequence
  Description of Artificial Sequence Synthetic peptide
DDDDDDPWQAYLDLFPTDTLALDLW

SEQ ID NO: 197 - protein, 23 aa, Artificial Sequence
  Description of Artificial Sequence Synthetic peptide
EEQQPWAQYLELLFPTETLLLEW

SEQ ID NO: 198 - protein, 23 aa, Artificial Sequence
  Description of Artificial Sequence Synthetic peptide
EEQQPWRAYLELLFPTETLLLEW

SEQ ID NO: 199 - protein, 23 aa, Artificial Sequence
  Description of Artificial Sequence Synthetic peptide
AEDQNPWARYADWLFPTTLLLLD

SEQ ID NO: 200 - protein, 23 aa, Artificial Sequence
  Description of Artificial Sequence Synthetic peptide
AEEQNPWARYAEWLFPTTLLLLE

SEQ ID NO: 201 - protein, 21 aa, Artificial Sequence
  Description of Artificial Sequence Synthetic peptide
AEDQNPWARYADLLFPTTLAW

SEQ ID NO: 202 - protein, 21 aa, Artificial Sequence
  Description of Artificial Sequence Synthetic peptide
AEEQNPWARYAELLFPTTLAW

SEQ ID NO: 203 - protein, 36 aa, Artificial Sequence
  Description of Artificial Sequence Synthetic polypeptide
DDDDDNPNYWARYANWLFTTPLLLLNGALLVEAEET

SEQ ID NO: 204 - protein, 36 aa, Artificial Sequence
  Description of Artificial Sequence Synthetic polypeptide
DDDDDNPNYWARYAPWLFTTPLLLLPGALLVEAEET

SEQ ID NO: 205 - protein, 34 aa, Artificial Sequence; identical to SEQ ID NO: 53
  Description of Artificial Sequence Synthetic polypeptide

SEQ ID NO: 206 - protein, 34 aa, Artificial Sequence; identical to SEQ ID NO: 51
  Description of Artificial Sequence Synthetic polypeptide

SEQ ID NO: 207 - protein, 20 aa, Artificial Sequence
  Description of Artificial Sequence Synthetic peptide
AEQNPIYWARYADWLFTTPL

SEQ ID NO: 208 - protein, 22 aa, Artificial Sequence
  Description of Artificial Sequence Synthetic peptide
AEQNPIYFARYADLLFPTTLAW

SEQ ID NO: 209 - protein, 22 aa, Artificial Sequence
  Description of Artificial Sequence Synthetic peptide
AEQNPIYWARYADLLFPTTLAF

SEQ ID NO: 210 - protein, 22 aa, Artificial Sequence
  Description of Artificial Sequence Synthetic peptide
AEQNPIYWARYADLLFPTTLAW

SEQ ID NO: 211 - protein, 20 aa, Artificial Sequence
  Description of Artificial Sequence Synthetic peptide
AEQNPIYFARYADWLFTTPL

SEQ ID NO: 212 - protein, 20 aa, Artificial Sequence
  Description of Artificial Sequence Synthetic peptide
EDQNPWARYADLLFPTTLAW

SEQ ID NO: 213 - protein, 34 aa, Artificial Sequence
  Description of Artificial Sequence Synthetic polypeptide
GLAGLAGLLGLEGLLGLPLGLLEGLWLGLELEGN

SEQ ID NO: 214 - protein, 32 aa, Artificial Sequence
  Description of Artificial Sequence Synthetic polypeptide
EQNPIYILDLVFGLLFAVTSVDFLVQWDDAGD

SEQ ID NO: 215 - protein, 24 aa, Artificial Sequence
  Description of Artificial Sequence Synthetic peptide
NLEGFFATLGGEIALWSLVVLAIE

SEQ ID NO: 216 - protein, 24 aa, Artificial Sequence
  Description of Artificial Sequence Synthetic peptide
NNEGFFATLGGEIALWSDVVLAIE

SEQ ID NO: 217 - protein, 25 aa, Artificial Sequence
  Description of Artificial Sequence Synthetic peptide
DNNEGFFATLGGEIPLWSDVVLAIE